(19) United States
(12) Patent Application Publication
(10) Pub. No.: US 2007/0100276 A1
(43) Pub. Date: May 3, 2007

(54) Apparatus and methods for clearing obstructions from surgical cutting instruments

(76) Inventors: Gary S. Fanton, Portola Valley, CA (US); John Krumme, Woodside, CA (US); John Peery, Santa Clara, CA (US); John E. Ashley, San Francisco, CA (US)

Correspondence Address: MEYERTONS, HOOD, KIVLIN, KOWERT & GOETZEL, P.C., 700 LAVACA, SUITE 800, AUSTIN, TX, 78701, US

(21) Appl. No.: 11/590,243

(22) Filed: Oct. 30, 2006

Related U.S. Application Data
(63) Continuation of application No. 11/359,885, filed on Feb. 21, 2006.
Application No. 11/359,885 is a continuation-in-part of application No. 11/187,604, filed on Jul. 21, 2005.
Application No. 11/187,604 is a continuation-in-part of application No. 10/782,489, filed on Feb. 18, 2004.

(51) Int. Cl. A61B 17/20

(52) U.S. Cl. 604/22

(57) ABSTRACT

The present invention relates to apparatus and methods for facilitating removal of obstructions from a surgical cutting instrument during a surgical procedure. The apparatus of the present invention is configured to interrupt flow in aspiration tubing when obstructions are detected in the cutting instrument. The apparatus then causes compression of the aspiration tubing to flush fluid towards the cutting instrument, thereby unclogging the instrument in a fast and efficient manner without the need to remove the instrument from the surgical site. A flush lumen is provided to flush a gas through the suction lumen to purge the material obstructing the suction lumen.

### REFERENCE TO RELATED APPLICATION

The present application is a continuation of U.S. patent application Ser. No. 11/359,885, filed Feb. 21, 2006, which is a continuation-in-part of U.S. patent application Ser. No. 11/187,604, filed Jul. 7, 2005, which is a continuation-in-part of U.S. patent application Ser. No. 10/782,489, filed Feb. 18, 2004. The above referenced applications are commonly owned with the present application, and the contents thereof are hereby incorporated by reference in their entirety as though fully set forth herein.

### BACKGROUND

1. Field of the Invention

The present invention generally relates to systems, methods and apparatus for clearing obstructions from surgical cutting instruments. More particularly, the present invention generally relates to medical devices which use suction to remove detritus from arthroscopic shaving and tissue cutting devices during an arthroscopic procedure without the removal thereof from the incision.

2. Description of the Related Art

Surgical cutting instruments such as mechanical shaving systems or microdebriders are well known for use in treating injured tissue in various bodily locations, such as joints. Many conventional cutting instruments operate by continuously rotating or by reciprocal rotation of a cutting edge. Such cutting instruments may be used in conjunction with the provision of irrigation fluid to the surgical site, and with the provision of a suction source to aspirate cut bodily tissue and irrigation fluid from the surgical site. The suction source also serves to draw tissue to the cutting edge before the tissue is debrided.

A common problem associated with conventional cutting instruments is clogging of the opening of the cutting edge from tissue that has not been cleanly severed, or is too large in diameter to fit through the opening in the cutting edge. The clogged cutting instrument must be removed from the arthroscopic site and the suction has to be stopped. Then, a physician or assistant has to manually clear the obstructing detritus from the cutting instrument in order to proceed with the surgical procedure. Many times, the physician is unable to remove obstructing matter from the instrument and must use a new instrument to continue surgery. Accordingly, clogging of such cutting instruments can cause a significant time delay in arthroscopic surgery and also result in additional costs due to the use of additional cutting instruments.

Previous inventions have attempted to reduce or eliminate problems associated with clogging of surgical cutting instruments. For example, U.S. Pat. No. 5,782,795 to Bays (Bays) describes a surgical suction cutting instrument with internal irrigation. The apparatus comprises an outer tubular member and an inner tubular member rotatably received in the outer tubular member. The distal end of the inner tubular member forms a cutting edge, and an aspiration lumen is formed within the inner tubular member. Further, an elongate tubular member is attached to the outer tubular member and configured to supply irrigation fluid to the cutting edge of the instrument.

During operation, the rotating cutting edge of the inner member engages and debrides tissue, and irrigation fluid from the elongate tubular member is provided to the cutting chamber. The debrided tissue and irrigation fluid are continuously removed through the aspiration lumen of the inner tubular member. By supplying irrigation fluid to the cutting chamber, the fluid can flush tissue and reduce clogging in the cutting chamber.

Apparatus and methods for rapidly and efficiently removing obstructing matter from a surgical cutting instrument are desirable.

Apparatus and methods for removing obstructing matter from a surgical cutting instrument without removing the instrument from the surgical site, thereby saving operating time, are further desired.

It is also desired to provide apparatus and methods for removing obstructing matter from a surgical cutting instrument within the surgical site without having to turn off a suction device coupled to the cutting instrument.

It yet further desired to provide apparatus and methods for removing obstructing matter from a surgical cutting instrument that can be used in conjunction with existing surgical suction cutting instruments.

### SUMMARY OF THE INVENTION

In view of the foregoing, it is an object of the present embodiments to provide an apparatus and methods for rapidly and efficiently removing obstructing matter from a surgical cutting instrument.

It is also an object of the present embodiments to provide apparatus and methods for removing obstructing matter from a surgical cutting instrument without removing it from the surgical site, thereby saving operating time.

It is a further object of the present embodiments to provide apparatus and methods for removing obstructing matter from a surgical cutting instrument within the surgical site without having to turn off a suction device coupled to the cutting instrument.

It is still a further object of the present embodiments to provide apparatus and methods for removing obstructing matter from a surgical cutting instrument that can be used in conjunction with existing surgical suction cutting instruments.

These and other objects of the present embodiments may be accomplished by providing apparatus comprising means for interrupting and means for flushing. The means for interrupting may be configured to interrupt aspiration flow in tubing coupled to the cutting instrument, while the means for flushing may be situated distal to the means for interrupting and configured to flush fluid in the tubing in a distal direction. The means for flushing urges fluid distally, towards the surgical site, to flush obstructions away from the cutting instrument. The term fluid may, of course, refer to either a gas or liquid.

In an embodiment, methods and devices for clearing obstructions from an aspiration lumen of a medical device, such as a cutting or shaving device, may include an outer tube and an inner tube having a cutting element disposed at the distal end of the inner tube, and an aspiration lumen extending through the inner tube.

In an embodiment, a flush lumen may be coupled to the aspiration lumen such that an obstruction may be cleared by delivering a gas through the aspiration lumen. A valve may be included to separate the flush lumen from the aspiration lumen until the valve is opened when a user operates the actuator. The valve may be open for a short time so that the area being treated is not overly inflated. In some embodiments, the valve may be open for less than 0.5 second or less than 0.3 second. The volume of gas passing through the open valve may be less than 500 ml to prevent excessive inflation of the area being treated.

In an embodiment, a gas holding chamber may be provided which holds the gas that will be used to clear the aspiration lumen when the valve is opened. The gas holding chamber may be adapted such that it expands when gas is provided thereto collapses when gas leaves the chamber. The elastic expansion of the gas holding chamber may be adapted such that the chamber urges the gas through the flush lumen.

In an embodiment, a flow element may be used to regulate delivery of the gas from a source of pressurized gas to the gas holding chamber. The flow element may be any suitable element, including a valve, or a fixed flow element. A fixed flow element may be sized to fill the gas holding chamber relatively slowly compared to the rate at which gas leaves the chamber when flushing the aspiration lumen. A flow element may provide direct pressure communication between the gas holding chamber and the source of pressurized gas.

Still further embodiments of the present invention are disclosed. Each embodiment is based on the principle of first interrupting aspiration in a lumen of the mechanical shaver handle and/or a lumen of the inner cutting member coupled to the mechanical shaver handle. Then, using a means for flushing, fluid situated distal to the means for interrupting is flushed in a distal direction to flush away obstructions. Alternative embodiments of the present invention based on these principles are described in detail herein below.

### BRIEF DESCRIPTION OF THE DRAWINGS

The above brief description as well as further objects, features and advantages of the methods and apparatus of the present invention will be more fully appreciated by reference to the following detailed description of presently preferred but nonetheless illustrative embodiments in accordance with the present invention when taken in conjunction with the accompanying drawings in which:

FIG. 1 is a schematic of a hypothetical surgical cutting instrument that may be used in conjunction with apparatus of the present invention;

FIGS. 2A-2B are, respectively, a side view and a side sectional view of a first embodiment of the present invention, which may be used to facilitate removal of obstructions from the cutting instrument of FIG. 1;

FIGS. 3A-3C are, respectively, side sectional views of the apparatus of FIGS. 2A-2B in fluid aspirating, interrupting and flushing states;

FIGS. 4A-4C are, respectively, side sectional views of an alternative embodiment of the apparatus of FIGS. 2-3 in fluid aspirating, interrupting and flushing states;

FIGS. 5A-5D are side views of a further alternative embodiment of the present invention in an aspirating state, fluid interrupting states, and a flushing state;

FIGS. 6A-6C are, respectively, side sectional views of an alternative embodiment of the present invention in fluid aspirating, interrupting and flushing states;

FIGS. 7A-7C are, respectively, a side view of a further alternative embodiment of the present invention in a fluid aspirating state, and side sectional views of the apparatus in fluid interrupting and flushing states;

FIG. 8 is an alternative embodiment of the invention described in FIGS. 7A-7C;

FIG. 9 is an alternative embodiment of the present invention showing means for interrupting and means for flushing disposed within a handle of a surgical cutting instrument;

FIG. 10 is an alternative embodiment of the apparatus of FIG. 9;

FIG. 11 is a side view of two components of a conventional disposable shaver blade set;

FIG. 12 is a side view depicting the two components of FIG. 11 in an assembled state;

FIG. 13 is a side view of a disposable shaver blade set provided in accordance with an embodiment;

FIGS. 14A-14B are, respectively, a side view and a front view of a housing configured for use with the disposable shaver blade set of FIG. 13;

FIG. 15 is a side-sectional view depicting the housing of FIG. 14;

FIGS. 16A-16C are, respectively, side views depicting the housing of FIG. 14 used in conjunction with the disposable shaver set of FIG. 13, and two alternative embodiments thereof;

FIGS. 17A-17B are, respectively, a side view and a side-sectional view of an alternative embodiment of FIG. 16;

FIGS. 18A-18C are, respectively, side-sectional views of a further alternative embodiment of the present invention in a fluid aspirating state, a fluid interrupting state and a fluid flushing state;

FIGS. 19A-19B are, respectively, a side views of an alternative embodiment and a side view of a means for flushing configured for use with the present invention;

FIG. 20 is a side-sectional view illustrating the components shown in FIG. 19A;

FIGS. 21A-21B are, respectively, side-sectional views of a further alternative embodiment in a fluid interrupting state and a fluid flushing state;

FIGS. 22A-22C are, respectively, side-sectional views of a further alternative embodiment in a fluid aspirating state, a fluid interrupting state and a fluid flushing state;

FIG. 23 is a plan view of one embodiment;

FIG. 24 is a plan view of an alternative embodiment;

FIG. 25 is a plan view of another alternative embodiment;

FIG. 26 is a plan view of yet another alternative embodiment;

FIG. 27 is a cross-sectional view of an embodiment; and

FIG. 28 is a plan view of yet another embodiment.

While the invention is susceptible to various modifications and alternative forms, specific embodiments thereof are shown by way of example in the drawings and will herein be described in detail. It should be understood, however, that the drawing and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

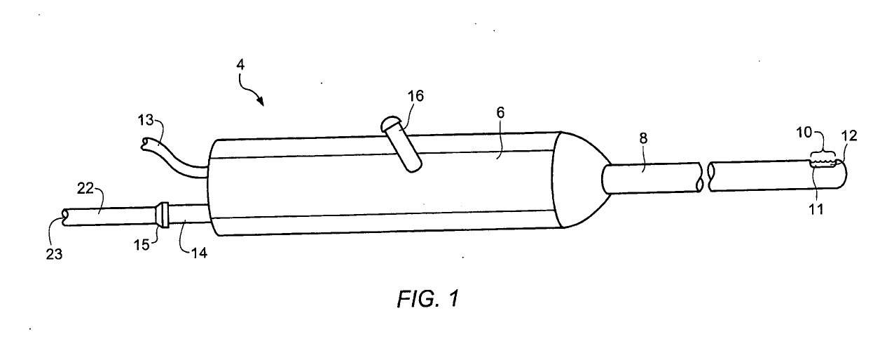

Referring to FIG. 1, a surgical suction cutting instrument that may be used in conjunction with apparatus of the present invention is described. Surgical suction cutting instrument 4 may be any conventional cutting instrument known in the art, and the particular features depicted in FIG. 1 are provided merely for illustrative purposes. In the embodiment depicted in FIG. 1, cutting instrument 4 comprises handle 6 having proximal and distal ends, and further comprises outer shaft 8 having proximal and distal ends. The proximal end of outer shaft 8 is coupled to the distal end of handle 6, while the distal end of outer shaft 8 comprises opening 10, as depicted in FIG. 1.

Outer shaft 8 houses inner shaft 11 having proximal and distal ends. The proximal end of inner shaft 11 is coupled to a motor (not shown), which is disposed in handle 6 and configured to drive rotation of cutting blade 12 on the distal end of shaft 11 when actuation means 16 is actuated. As will be apparent to those skilled in the art, opening 10 is configured to permit tissue to be cut by rotation of cutting edge 12 with respect to outer shaft 8.

Referring still to FIG. 1, surgical suction cutting instrument 4 further comprises electrical supply means 13 and aspiration line 14 coupled to handle 6. Electrical supply means 13 is coupled to the motor, thereby driving rotation of cutting edge 12.

In FIG. 1, aspiration line 14 is coupled to aspiration tubing 22, which has proximal and distal ends. The proximal end of aspiration tubing 22 is coupled to a suction device (not shown), while the distal end of aspiration tubing 22 is coupled to aspiration line 14 via port 15. Alternatively, port 15 may be omitted, such that aspiration tubing 22 and aspiration line 14 are in effect the same line.

When the suction device is turned on, suction is provided through lumen 23 of aspiration tubing 22, which is in fluid communication with the distal end of outer shaft 8. Accordingly, suction is provided to cutting edge 12 and opening 10 to draw tissue towards the cutting edge, and also to facilitate removal of debrided tissue and irrigation fluid from a surgical site.

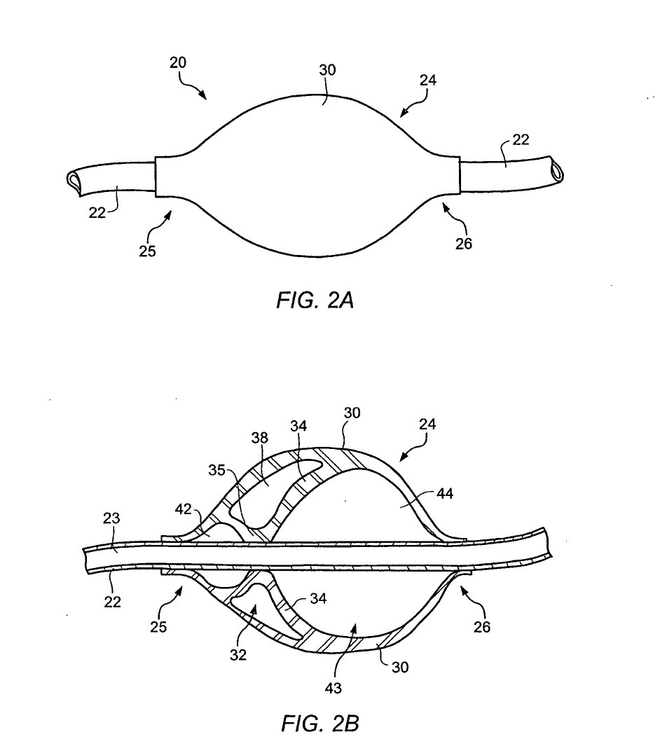

Referring now to FIGS. 2A-2B, a first embodiment of the present invention is described. Apparatus 20 is configured to facilitate removal of clogged tissue from a surgical cutting instrument, such as cutting instrument 4 of FIG. 1.

In an embodiment, the apparatus may include an actuation means having a preferably bulb-shaped exterior surface. The actuation means is configured to be useable with aspiration tubing coupled to the cutting instrument, such that the actuation means encloses the aspiration tubing during use. Apparatus 20 comprises actuation means 24 having proximal and distal regions 25 and 26, respectively, as shown in FIG. 2A. Actuation means 24 is configured to be used in conjunction with at least one piece of aspiration tubing 22 that is in fluid communication with cutting instrument 4, for example, as depicted in FIG. 1.

Actuation means 24 comprises exterior surface 30, which preferably is a bulb-shaped member that is adapted to be grasped by a human hand. Exterior surface 30 may be manufactured using a suitable compound, such as rubber, that allows the exterior surface to be compressed in an inward direction when a force is applied and then return to its original, non-compressed state when the compressive force is removed.

A means for interrupting may include at least one inward protrusion disposed between the bulb-shaped exterior surface and a section of the aspiration tubing. The means for flushing may include at least one fluid chamber disposed between the exterior surface and the aspiration tubing at a location distal to the means for interrupting. During use, the aspiration tubing may be configured to aspirate matter, such as debrided tissue and irrigation fluid, from a surgical site. When no external compressive forces are applied to the actuation means, the inward protrusion does not impose substantial forces upon the aspiration tubing, thereby allowing aspiration to be achieved throughout the tubing. When an obstruction is present in the cutting instrument, the bulb-shaped exterior of the actuation means may be compressed to cause the inward protrusion to compress a section of aspiration tubing. This inhibits aspiration in the tubing distal to the inward protrusion.

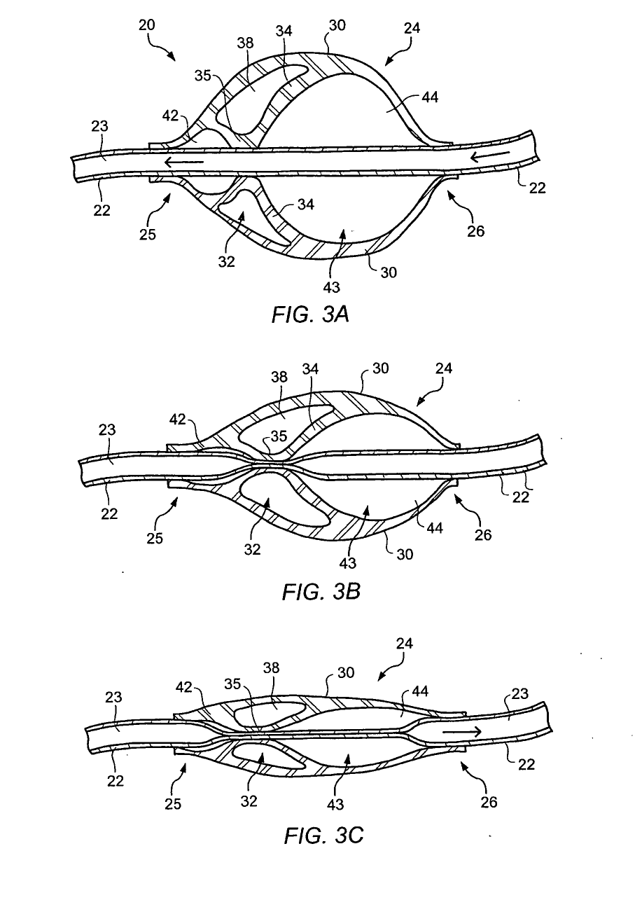

Apparatus 20 further includes means for interrupting 32, as shown in FIG. 2B. In the embodiment of FIGS. 2-3, means for interrupting 32 comprises at least one inward protrusion 34 that is disposed within exterior surface 30 and configured to selectively compress aspiration tubing 22. Inward protrusion 34 preferably has a curved shape with at least one apex or engagement point 35, as shown in FIG. 2B. Apex 35 of inward protrusion 34 is configured to selectively compress an exterior surface of aspiration tubing 22, and may fully encircle tubing 22, contact the tubing at one location, contact the tubing at two opposing locations, or contact the tubing in any other manner suitable for applying compressive force upon tubing 22.

If apex 35 fully encircles tubing 22, then the provision of a bulb-shaped exterior surface 30 may be desirable. However, where two opposing apexes are provided, it may be desirable to provide exterior surface 30 with indicia (not shown) corresponding to the circumferential positioning of apexes 35 beneath exterior surface 30. Such indicia allows a user to compress exterior surface 30 at a location that will most effectively cause compression of the apexes to actuate the device, as described hereinbelow. Alternatively, when two opposing apexes 35 are employed, exterior surface 30 may comprise an elliptical or oval shape, so that a physician can simply compress the opposing surfaces to effectively cause compression of the apexes.

Inward protrusion 34 may be formed from the same material as exterior surface 30 of actuation means 24, or alternatively, may be manufactured using a separate material that is bonded to an interior region of exterior surface 30. If a separate material is used, it may be desirable to provide inward protrusion 34 as a more rigid member than exterior surface 30, so that compression of exterior surface 30 will result in a stronger and more direct compression of tubing 22.

Apparatus 20 may comprise at least one proximal fluid chamber 42 formed between aspiration tubing 22 and exterior surface 30, at a location proximal to apex 35. Further, recess 38 may be formed between inward protrusion 34 and exterior surface 30, as depicted in FIG. 2B. Alternatively, these regions may be solid, such that recess 38 and/or fluid chamber 42 are omitted entirely.

Referring still to FIG. 2B, apparatus 20 further comprises means for flushing 43, which in the embodiment of FIGS. 2-3 comprises at least one fluid chamber 44 formed between aspiration tubing 22 and exterior surface 30. Fluid chamber 44 of means for flushing 43 is disposed at a location distal to apex 35, as shown in FIG. 2B.

Referring now to FIGS. 3A-3C, use of apparatus 20 of FIGS. 2A-2B is described for facilitating removal of clogged tissue from cutting instrument 4 during a surgical procedure, such as arthroscopic surgery. As will be apparent to one skilled in the art, irrigating fluid may be delivered to the surgical site, while aspiration tubing 22 is employed to aspirate irrigating fluid and cut tissue from the surgical site. An irrigation line (not shown) may be used in conjunction with apparatus 20 of the present invention, if desired.

In FIG. 3A, actuation means 24 is shown in an “open” or fully aspirating state. The proximal end of aspiration tubing 22 is coupled to a suction device, and aspiration tubing 22 is in fluid communication with the distal end of cutting instrument 4. Accordingly, irrigation fluid and cut tissue from the surgical site will be aspirated through opening 10 of cutting instrument 4, and then through tubing 22 in a proximal direction, as indicated by the arrows in FIG. 3A.

As noted above, one common problem associated with use of conventional surgical cutting instruments during arthroscopic procedures is the tendency of cut tissue to clog opening 10 or cutting edge 12 of the cutting instrument. In accordance with one aspect of the present invention, once the cutting instrument becomes clogged, a physician may manually compress exterior surface 30 to cause apex 35 to compress a section of aspiration tubing 22, as shown in FIG. 3B. This inhibits aspiration distal to apex 35, such that no substantial fluid flow occurs in tubing 22 distal to apex 35.

Further compression of the bulb-shaped exterior compresses the fluid chamber of the flushing means. This causes compression of a section of tubing distal to the means for interrupting. Fluid in the aspiration tubing, distal to the means for interrupting, then is flushed in a distal direction. By causing fluid in the aspiration tubing to flow in a distal direction, the fluid flushes clogged tissue away from the cutting instrument. Referring now to FIG. 3C, when a physician applies further compression to actuation means 24, means for flushing 43 is actuated. Specifically, the compression of exterior surface 30 towards tubing 22 causes fluid in chamber 44 to compress tubing 22. The compression of tubing 22 causes irrigation fluid in tubing 22 that is distal to apex 35 to be urged in a distal direction, i.e., towards opening 10 and cutting edge 12. Since apex 35 remains sealingly engaged with aspiration tubing 22, fluid in tubing 22 will be flushed in a distal direction.

By applying pressure to fluid in tubing 22 and flushing fluid in a distal direction, the distally flowing fluid flushes clogged tissue away from opening 10 and cutting edge 12 of the cutting instrument. Once compression is released, this tissue then may be introduced back into the cutting instrument, reduced in size, and then effectively aspirated through tubing 22.

In an embodiment, and in accordance with principles described herein, the cutting instrument need not be removed from the surgical site, or a replacement instrument need not be provided thereto, because the obstructing tissue is effectively removed at the surgical site. Further, the suction device need not be stopped to remove the obstruction, since aspiration is blocked by the means for interrupting. The surgical procedure need therefore not be substantially delayed, nor equipment need be replaced.

Advantageously, in accordance with one aspect of the present invention, a physician may remove obstructing tissue from the surgical cutting instrument using actuation means 24 without having to remove the cutting instrument from the surgical site and manually remove the clogged tissue. Also, the surgeon is not expected to have to replace the cutting instrument with a different instrument, thereby saving time and money.

Upon successful removal of clogged tissue, the physician can remove the compressive forces imposed upon actuation means 24, thereby causing actuation means 24 to return to its original shape, depicted in FIG. 3A. At this time, aspiration throughout the tubing is restored.

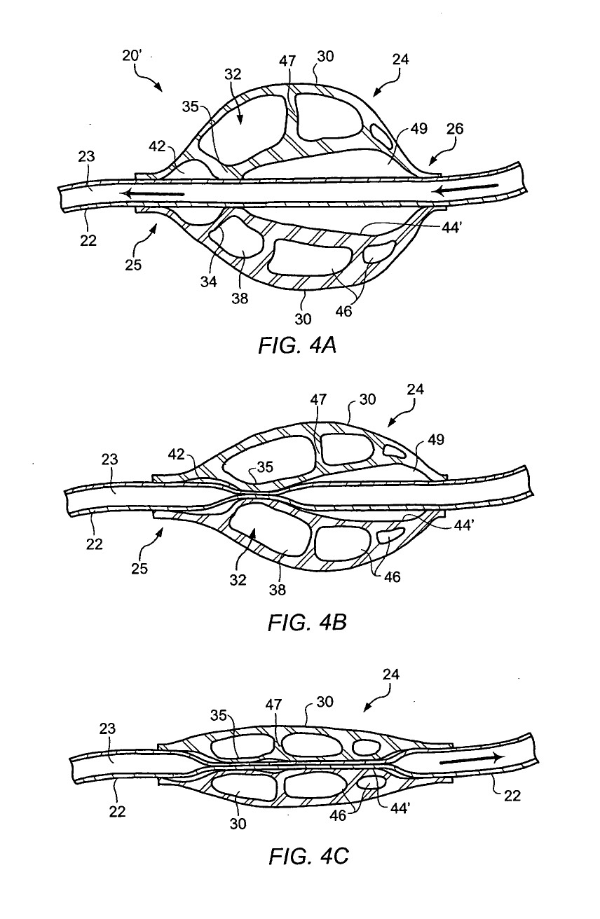

Referring now to FIGS. 4A-4C, an alternative embodiment of apparatus 20 of FIGS. 2-3 is described. Apparatus 20′ is similar to apparatus 20, except as noted hereinbelow. In particular, an alternative means for flushing, comprising at least one interior compression member 44′, is employed. Interior compression member 44′ is configured to apply a direct compressive force to aspiration tubing 22, as described hereinbelow.

Apparatus 20′ preferably further comprises at least one support structure 47 disposed between exterior surface 30 and interior compression member 44′, as shown in FIG. 4A. Fluid chambers 46 may be formed between support structures 47, exterior surface 30, and/or interior compression member 44′. Support structures 47 help translate compressive forces from exterior surface 30 to interior compression member 44′, while fluid chambers 46 conform to provide flexibility and comfort during operation.

The operation of apparatus 20′ is similar to use of apparatus 20, as described in FIGS. 3A-3C hereinabove. In a first step, actuation means 24 is provided in an “open” or fully aspirating state, as shown in FIG. 4A. Once cutting instrument 4 becomes clogged, a physician may manually compress exterior surface 30 of actuation means 24 to cause apex 35 of means for interrupting 34 to sealingly compress a section of aspiration tubing 22, as shown in FIG. 4B. This inhibits aspiration distal to apex 35.

Referring now to FIG. 4C, apparatus 20′ is shown when a physician applies further compression to exterior surface 30 of actuation means 24. The compression of exterior surface 30 towards tubing 22 causes support structures 47 to translate the compressive force to interior compression member 44′, which in turn directly compresses a portion of tubing 22 distal to apex 35. The direct compression of tubing 22 causes irrigation fluid in tubing 22 that is distal to apex 35 to be urged in a distal direction, i.e., towards opening 10 and cutting edge 12. As described hereinabove, by applying pressure to fluid in tubing 22 and causing the fluid to flow in a distal direction, the fluid flushes clogged tissue away from the cutting edge of the instrument.

In the embodiments of FIGS. 2-4 hereinabove, it will be apparent to one skilled in the art that either air or liquid may be disposed within chambers 38, 42, 44, 46 and 49. In particular, the provision of liquid in chambers 44, 46 and 49 is expected to facilitate compression of tubing 22. Alternatively, any of the fluid chamber depicted hereinabove may be omitted and replaced with solid regions.

In an alternative embodiment, fluid trapped in chambers 44 of FIGS. 3A-3C may be delivered to aspiration tubing 22 to facilitate removal of clogged tissue in the cutting instrument. In this embodiment, a section of tubing 22, situated between apex 35 and distal region 26, may comprise a plurality of small perforations (not shown). In the step described in FIG. 3C, fluid disposed in chamber 44 may be infused into tubing 22 via the plurality of small perforations. The infused fluid then will flow in a distal direction through lumen 23 to facilitate removal of clogged tissue. Similarly, for the embodiment described in FIGS. 4A-4C, perforations may be provided in tubing 22 and liquid disposed in chambers 49 may be infused into lumen 23 upon compression of exterior surface 30.

Further, as will be apparent to one skilled in the art, varying degrees of aspiration tubing stiffness may be provided. For example, relatively flexible aspiration tubing 22 may be provided when air is disposed in chambers 44 and 49, to ensure that the air may compress the tubing. By contrast, relatively rigid aspiration tubing may be provided when liquid is disposed in chambers 44 and 49 to facilitate compression of the relatively stiff tubing. In any embodiment, tubing 22 also may comprise at least one relatively flexible segment and at least one relatively rigid segment.

In accordance with another aspect of the present invention, it should be noted that apparatus 20 may be used in conjunction with any existing cutting instrument 4. Apparatus 20 may be provided securely disposed about tubing 22, or alternatively, apparatus 20 may be provided as a separate component. In the latter case, apparatus 20 may slide over tubing 22 and a user may secure proximal and distal regions 25 and 26 to tubing 22 at a desired location on the tubing. For example, apparatus 20 may be secured about tubing 22 using a suitable adhesive, thermal plastic bond, or using mechanical means such as clamps. A physician therefore may vary the longitudinal positioning of apparatus 20 with respect to tubing 22. However, it may be desirable to have apparatus 20 disposed towards the distal end of tubing 22 so that the apparatus is in relatively close proximity to cutting instrument 4. In an embodiment, an actuation means may include first and second handles configurable to actuate first and second rollers. The first and second rollers are configured to move within respective channels in the handles. The first an second rollers may be configured to serve both as the means for interrupting aspiration and the means for flushing fluid in the aspiration tubing.

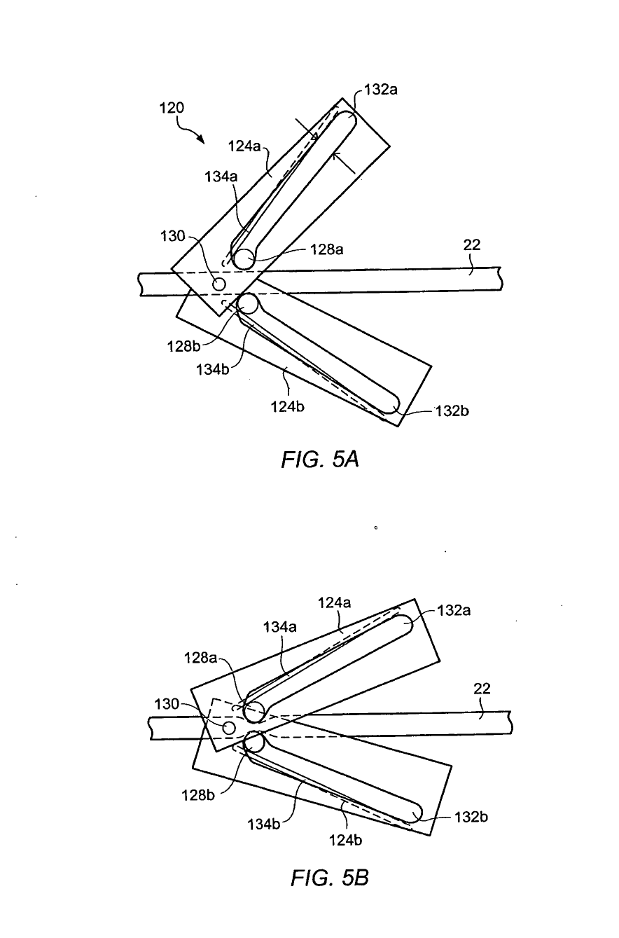

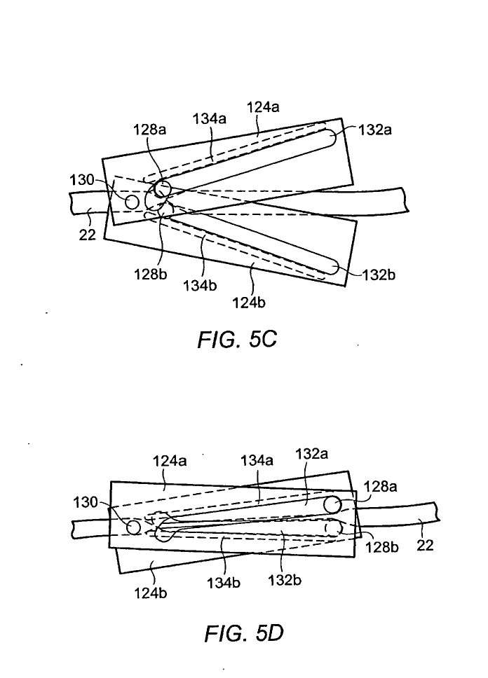

Referring now to FIGS. 5A-5D, an alternative embodiment of the present invention is described for facilitating removal of clogged tissue during a surgical procedure. Apparatus 120 comprises an actuation means having first handle 124a and second handle 124b. First handle 124a has channel 132a disposed therein, while second handle 124b has channel 132b disposed therein, as shown in FIG. 5A. First and second handles 124a and 124b are coupled together at their respective proximal ends using pivot pin 130, which is disposed to partially or fully surround aspiration tubing 22.

Apparatus 120 preferably further comprises springs 134a and 134b, each having proximal and distal ends. The proximal ends of springs 134a and 134b preferably are coupled to the proximal ends of handles 124a and 124b, respectively, while the distal ends of springs 134a and 134b are coupled to the distal ends of handles 124a and 124b, respectively, as shown in FIG. 5A. Alternatively, the proximal ends of the springs may be left unsecured, so that the proximal ends of the springs may deflect when the device is actuated, as described further in FIGS. 5C-5D hereinbelow.

Apparatus 120 further comprises means for interrupting and means for flushing. In the embodiment depicted herein, both the means for interrupting and means for flushing are the same, and they comprise first and second rollers 128a and 128b. First and second rollers 128a and 128b preferably comprise an outer diameter that is slightly smaller than height h of channels 132a and 132b, thereby permitting the rollers to move longitudinally within their respective channels, as described hereinbelow.

In an aspirating state, the rollers are disposed at a proximal section of their respective channels, and do not impose substantial forces upon the aspiration tubing. When obstructing tissue is present at the cutting edge of the instrument, the handles may be compressed to advance the rollers distally within the channels. As compression is applied aspiration flow in the tubing may be interrupted. Then, the rollers may be urged distally to distally advance the fluid in the tubing distal to the rollers. The distally urged fluid flushes the surgical site and facilitates removal of clogged tissue from the cutting instrument. Referring still to FIG. 5A, apparatus 120 is depicted in an “open” or fully aspirating state, whereby the distal ends of handles 124a and 124b are widely separated. In the aspirating state, rollers 128a and 128b are disposed in proximal sections of their respective channels. Rollers 128a and 128b do not apply substantial forces to tubing 22 in the fully aspirating state.

Referring now to FIG. 5B, a physician may realize cutting instrument 4 has become clogged during a surgical procedure. When this occurs, the physician applies a compressive force to first and second handles 124a and 124b. The compressive force causes the proximal ends of the handles to rotate about pivot point 130, and further causes the distal ends of the handles to be drawn closer together, as depicted in FIG. 5B.

As handles 124a and 124b are compressed together, rollers 128a and 128b apply a compressive force upon aspiration tubing 22, as shown in FIG. 5B. The compressive force of the rollers pinches the tubing and inhibits further aspiration of fluid in lumen 23 (distal to the rollers). When relatively light compressive forces are applied to handles 124a and 124b, springs 134a and 134b do not substantially displace, and therefore serve to confine rollers 128a and 128b at the proximal sections of their respective channels.

Referring now to FIG. 5C, as a physician further compresses handles 124a and 124b, proximal regions of springs 134a and 134b are displaced against the rollers. Displacement of springs 134a and 134b, above a predetermined compression threshold, causes rollers 128a and 128b to advance distally within their respective channels.

Referring now to FIG. 5D, still further compression of handles 124a and 124b causes rollers 128a and 128b to be advanced towards the distal end of channels 132a and 132b, respectively. Rollers 128a and 128b remain engaged with tubing 22, such that their advancement flushes fluid in lumen 23, situated distal to the rollers, in a distal direction.

By applying pressure to fluid in tubing 22 and flushing the fluid in a distal direction, the fluid flow flushes clogged tissue from opening 10 and cutting edge 12. Upon successful removal of clogged tissue, the physician can remove the previously-applied compressive forces imposed upon handles 124a and 124b, thereby causing the handles to return to their original positions, depicted in FIG. 5A.

As will be apparent to one skilled in the art, characteristics of springs 134a and 134b may be varied to vary the manual force required to actuate apparatus 120. However, the springs preferably are stiff enough so that they do not substantially displace until tubing 22 first is compressed. After a threshold force is applied to compress tubing 22, then the springs are configured to displace enough to permit rollers 128a and 128b to be advanced distally in channels 132a and 132b.

Further, the distance between pivot point 130 and the proximal ends of channels 132a and 132b may be varied to vary the force required to actuate apparatus 120. For example, increasing this distance may reduce the force required to actuate apparatus 120, since greater leverage is provided.

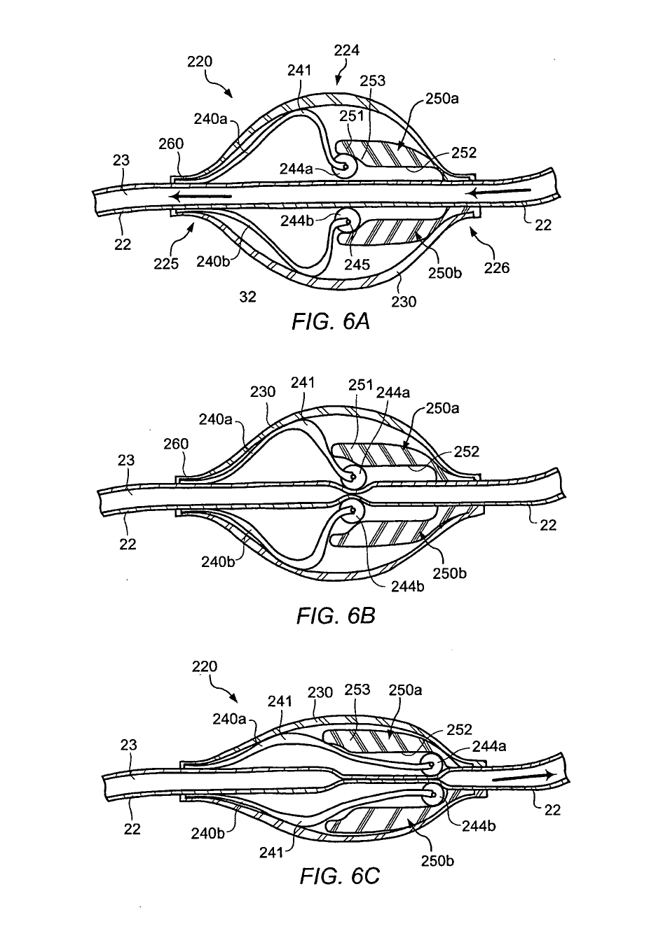

In an embodiment an actuation means having a preferably bulb-shaped exterior surface and at least one arcuate spring disposed within the exterior surface may be employed. A roller may be coupled to a distal end of each arcuate spring, and the rollers may be configured to advance within roller guides disposed within the actuation means. Referring now to FIGS. 6A-6C, a further alternative embodiment of the present invention is described for clearing obstructions from cutting instrument 4. Apparatus 220 comprises actuation means 224, which comprises exterior surface 230. Apparatus 220 further comprises means for interrupting and means for flushing, which, in the embodiment of FIGS. 6A-6C, are the same. The means for interrupting and the means for flushing comprise first and second arcuate springs 240a and 240b, and further comprise rollers 244a and 244b.

Arcuate springs 240a and 240b have proximal and distal ends and a central region 241 disposed therebetween. The proximal ends of arcuate springs 240a and 240b are fixedly attached to an interior portion of exterior surface 230 at fixation point 260, as shown in FIG. 6A. The distal ends of arcuate springs 240a and 240b are coupled to rollers 244a and 244b, respectively. Rollers 244a and 244b preferably are coupled to their respective springs using a central pin 245, as depicted in FIG. 6A.

In an embodiment, the bulb-shaped exterior may be compressed during use to compress a central region of the arcuate spring. Compression of the spring urges the rollers by way of the roller guides inwardly to compress the aspiration tubing and interrupt aspiration flow.

When no external forces are applied to apparatus 220, central regions 241 of arcuate springs 240a and 240b are configured to assume the curved configuration depicted in FIG. 6A. When compressive forces are applied, the arcuate springs may be deformed accordingly, as will be described in greater detail hereinbelow.

Apparatus 220 further comprises first and second roller guides 250a and 250b, each having proximal region 251 and distal region 252. Each proximal region 251 transitions into distal region 252 via curvature or slant 253. Each proximal region 251 is configured to house rollers 244a and 244b in the aspirating state, as depicted in FIG. 6A and described in greater detail hereinbelow.

In accordance with one aspect of the present invention, apparatus 220 is configured for use with existing surgical cutting instruments, such as cutting instrument 4 of FIG. 1. Apparatus 220 may be affixed to tubing 22, for example, at proximal and distal regions 225 and 226, or alternatively, apparatus 220 may slide longitudinally over the tubing. In the latter embodiment, a physician may insert the tubing through a central region of apparatus 220, and then position apparatus 220 at a desired location with respect to the tubing.

The operation of apparatus 220 is similar to use of apparatus 20, as described in FIGS. 3A-3C hereinabove. In a first step, apparatus 220 is provided in an “open” or fully aspirating state, whereby no substantial external forces are applied to apparatus 220. In this state, central regions 241 of arcuate springs 240a and 240b assume the curved configuration depicted in FIG. 6A. At this time, rollers 244a and 244b are disposed within proximal regions 251 of their respective roller guides 250a and 250b. Accordingly, rollers do not impose a substantial force upon tubing 22 during the aspirating state, as shown in FIG. 6A. As noted above, when the proximal end of tubing 22 is coupled to a suction device (not shown), irrigation fluid and cut tissue from the surgical site will be aspirated through aspiration tubing 22 in a proximal direction, as indicated by the arrows in FIG. 6A.

Referring now to FIG. 6B, once suction tubing 22 becomes clogged, a physician may manually compress exterior surface 230 to compress central region 241 of arcuate springs 240a and 240b. Compression of the arcuate springs causes rollers 244a and 244b to advance distally, since the proximal ends of the arcuate springs remain fixed at points 260.

When compression is applied, slant 253 urges rollers 244a and 244b in an inward direction, i.e., towards tubing 22, as shown in FIG. 6B. Rollers 244a and 244b sealingly compress a section of aspiration tubing 22, thereby inhibiting suction within lumen 23 (distal to the rollers).

Further compression of the bulb-shaped exterior of the actuation means may guide the rollers distally while pinching flow in the tubing. This flushes fluid in the aspiration tubing, situated distal to the rollers, in a distal direction to facilitate removal of obstructing tissue. Referring now to FIG. 6C, apparatus 220 is shown when a physician applies further compression to exterior surface 230 of actuation means 224. The compression of exterior surface 230 towards aspiration tubing 22 urges springs 240a and 240b to straighten, thereby advancing rollers 244a and 244b in a distal direction, as shown in FIG. 6C. The rollers are guided by distal region 252 of roller guides 250a and 250b, which ensure that the rollers remain firmly engaged with tubing 22. The advancement of rollers 244a and 244b causes irrigation fluid in tubing 22 that is distal to the rollers to be flushed in a distal direction, i.e., towards opening 10 and cutting edge 12, thereby flushing obstructions away from the cutting instrument.

Upon successful removal of clogged tissue, the surgeon can remove the previously-applied compressive forces, thereby causing springs 240a and 240b to return to their preferred arcuate shapes, as depicted in FIG. 6A. At this time, aspiration throughout the tubing is restored, and will remain in the aspirating state until the surgeon applies further compressive forces to remove subsequently clogging tissue.

As will be apparent to one skilled in the art, characteristics of springs 240a and 240b may be varied to vary the manual force required to actuate apparatus 220. However, the springs should be stiff enough to cause rollers 244a and 244b to compress tubing 22 without substantially bowing inward first, i.e., the springs will not merely bow inward before the tubing is displaced. After a threshold force is applied to compress tubing 22, then the springs are configured to displace enough to permit rollers 244a and 244b to be advanced distally in channels 250a and 250b.

Further, it will be apparent to one skilled in the art that, in lieu of two or more roller guides 250a and 250b, one continuous, circumferentially-shaped roller guide 250 may be disposed within exterior surface 230. Such a continuous and circumferential guide may be adapted to guide each roller 244. In such an embodiment, a bulb-shaped exterior surface 230 may be desirable.

However, where two rollers and two roller guides are employed, as depicted in FIGS. 6A-6C, it may be desirable to provide exterior surface 230 with indicia (not shown) corresponding to the circumferential positioning of springs 244a and 244b beneath exterior surface 230. Such indicia allows a user to compress exterior surface 230 at a location that will most effectively actuate springs 240a and 240b. Alternatively, when two springs and two roller guides are employed, exterior surface may comprise an elliptical or oval shape, so that a physician can simply compress the opposing regions to effectively actuate the device.

Finally, it will be apparent that although two rollers 244a and 244b are depicted, greater or fewer rollers may be employed to achieve the effects described hereinabove.

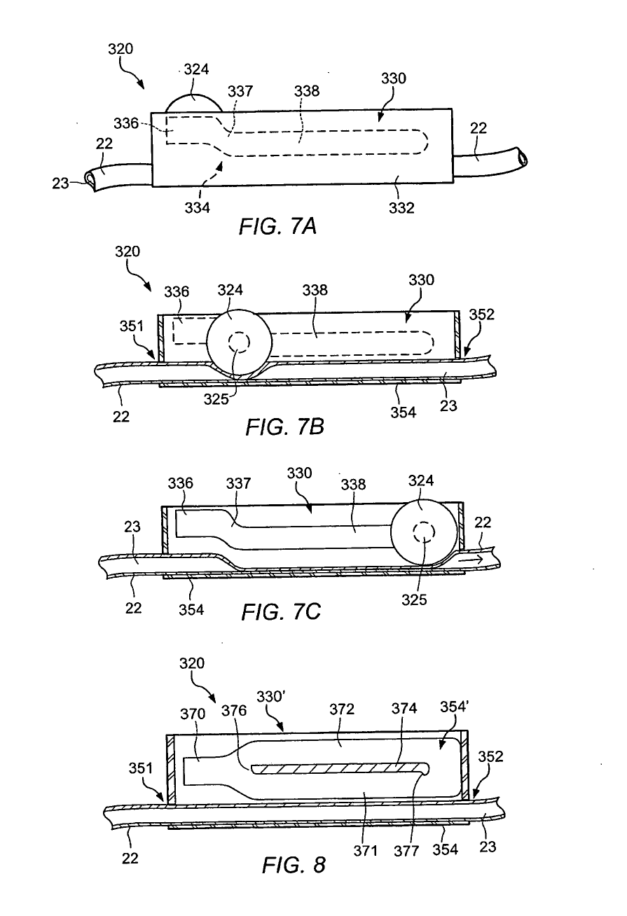

Referring now to FIGS. 7A-7C, a further alternative embodiment of the present invention is described. Apparatus 320 comprises roller 324 and housing 330, which are configured to be used in conjunction with aspiration tubing 22 having lumen 23 disposed therethrough. In this embodiment, roller 324 serves as the actuation means, and also as the means for interrupting and means for flushing, as will be described in greater detail hereinbelow.

Housing 330 preferably comprises a rectangular shape, although other shapes may be employed. Channel 334 having proximal region 336, central region 337 and distal region 338 is disposed within housing 330, as shown in FIG. 7A. In a preferred embodiment, distal region 338 spans a greater length than proximal region 336.

Roller 324 is coupled to roller axle 325, as shown in FIG. 7B. Roller axle 325 preferably has an outer diameter that is slightly smaller than height h′ of channel 334, thereby permitting roller 324 to be advanced longitudinally within channel 334 via roller axle 325.

Tubing 22 is disposed through proximal and distal openings 351 and 352 of housing 330, as depicted in FIG. 7B. The tubing may be inserted through the proximal and distal openings when roller 324 is situated in proximal region 336 of channel 334, as shown in FIG. 7A. Proximal and distal openings 351 and 352 preferably are disposed just above lower surface 354 of housing 330, so that tubing 22 can rest upon lower surface 354 during operation.

A physician may advance apparatus 320 longitudinally with respect to tubing 22 until housing 330 is disposed in a desired location with respect to the tubing. The physician then may secure the position of housing 330 with respect to tubing 22, e.g., using an adhesive or mechanical means, or leave the housing unsecured.

Referring to FIG. 7A, apparatus 220 is provided in an “open” or fully aspirating state, whereby roller 324 is disposed in proximal region 336 of channel 334. At this time, roller 324 is constrained at such a distance from tubing 22 that the roller does not impose a substantial compressive force upon the tubing. This permits aspiration through tubing 22 when the proximal end of the tubing is coupled to a suction device.

Referring now to FIG. 7B, once a cutting instrument coupled to tubing 22 becomes clogged during a surgical procedure, a physician may advance roller 324 distally, either manually or using mechanical means (not shown). It should be noted that roller 324 may be round, as depicted, or alternatively may comprise one or more grooves or raised surfaces to provide a frictional resistance that facilitates actuation by the physician.

As roller 324 is advanced distally, the contours of central region 337 cause roller axle 325 to direct roller 324 in an inward direction, i.e., towards tubing 22. Roller 324 subsequently pinches off a section of tubing 22 by compressing the tubing between the roller and lower surface 354 of housing 330, as shown in FIG. 7B. This advancement of roller 324 inhibits suction within lumen 23 (distal to the roller).

Referring now to FIG. 7C, apparatus 320 is shown when a physician further advances roller 324 distally. Roller axle 325 is guided along distal region 338 of channel 334. During this time, roller 324 applies continuous compression upon tubing 22, which is compressed against lower surface 354.

The advancement of roller 324 causes irrigation fluid in tubing 22 that is distal to roller 324 to be flushed in a distal direction, i.e., towards opening 10 and cutting edge 12. As described hereinabove, by applying pressure to fluid in tubing 22 and causing the fluid to flow in a distal direction, the fluid flushes clogged tissue away from cutting edge 12.

Upon successful removal of clogged tissue, a surgeon can retract roller 324 proximally into proximal region 336, thereby causing roller 324 to become fully or substantially disengaged from tubing 22. Tubing 22 will return to its uncompressed shape and aspiration will be restored throughout the tubing.

Referring now to FIG. 8, an alternative embodiment of apparatus 320 of FIGS. 7A-7C is described. In FIG. 8, apparatus 320′ functions similarly to apparatus 320, except as noted hereinbelow. Apparatus 320′ comprises housing 330′ having channel 334′. Channel 334′ comprises proximal section 370, advancement channel 371 and return channel 372, which preferably are separated by partition 374. Apparatus 320′ further comprises roller 324 of FIGS. 7A-7C, which is omitted from FIG. 8 for clarity.

In operation, roller 324 is positioned within proximal section 370 of channel 334′ in the aspirating state. Once the cutting instrument becomes clogged, a physician may advance roller 324 distally towards one-way guide 376 of partition 374. One-way guide 376 causes roller 324 to be advanced distally into advancement channel 371.

When roller 324 is advanced into advancement channel 371, roller 324 subsequently pinches off a section of tubing 22 by compressing the tubing between the roller and lower surface 351 of housing 330. At this time, roller 324 inhibits suction within lumen 23 (distal to the roller). As roller 324 is further advanced in advancement channel 371, irrigation fluid in tubing 22 that is distal to the roller is flushed in a distal direction, i.e., towards cutting edge 12.

When roller 324 is advanced toward the distal end of advancement channel 371, distal stop 377 of partition 374 may provide resistance to roller axle 325 (see FIGS. 7B-7C). A surgeon may apply a slightly greater force upon roller 324 to cause the roller axle to be advanced beyond distal stop 377.

Once the roller axle is advanced beyond distal stop 377, the roller is returned to proximal region 370 via return channel 372. In one embodiment, a physician may manually advance roller 324 in a proximal direction through return channel 372 and into proximal region 370. Alternatively, as will be apparent to one skilled in the art, a spring mechanism (not shown) may be employed to facilitate the return of roller 324 to proximal region 370, after the roller passes distal stop 377. It should be noted that, in either the manual or spring-return embodiments, distal stop 377 also may be omitted entirely.

With respect to all embodiments described hereinabove, it will be apparent to one skilled in the art that the means for interrupting and means for flushing may be completely separate entities, each actuated using its own separate actuation means. Specifically, the means for interrupting may compress a first section of tubing 22 to interrupt aspiration within the tubing. Then, the means for flushing, which is disposed at a second location along tubing 22 distal to the means for interrupting, subsequently may be actuated to cause irrigation fluid to be flushed towards cutting edge 12.

Additionally, in other embodiments, the means for flushing may be configured to be pressurized. For example, a desired amount of pressure may be built up in a bulb-shaped compressor. Once a desired pressure is reached, the means for flushing is actuated to permit pressurized fluid to compress tubing 22, thereby resulting in high velocity fluid flow through opening 10 of the cutting instrument. If desired, such pressurized means for flushing can be configured to automatically release fluid once a predetermined pressure threshold is achieved.

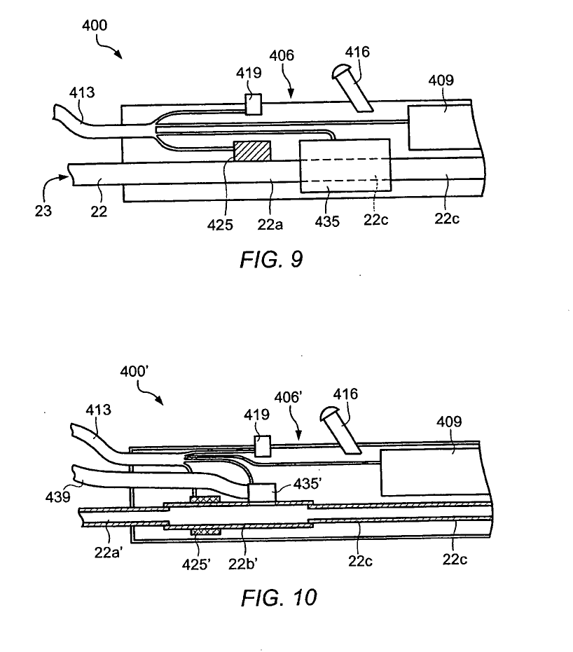

Referring now to FIGS. 9-10, further alternative embodiments of the present invention are described. The embodiments of FIGS. 2-8 described apparatus for facilitating removal of obstructions from a surgical cutting instrument, whereby the apparatus was disposed substantially or exclusively on a section of aspiration tubing external to the cutting instrument. In the embodiments of FIGS. 9-10, similar apparatus are described; however, these embodiments are substantially integrated into a handle of the cutting instrument itself.

Referring to FIG. 9, apparatus 400 comprises cutting instrument handle 406 having proximal and distal ends. Handle 406 is similar to handle 6 of cutting instrument 4 of FIG. 1, except as noted hereinbelow. Like the embodiment of FIG. 1, the distal end of handle 406 of FIG. 9 may be coupled to outer shaft 8 having distal opening 10 (see FIG. 1).

Apparatus 400 further comprises motor 409, which is disposed within handle 406 and configured to drive inner shaft 11 of FIG. 1. Electrical supply means 413 is coupled to handle 406, and is adapted to communicate with multiple components of apparatus 400, as described hereinbelow.

Aspiration tubing 22 having proximal and distal ends also is provided. The proximal end of aspiration tubing 22 is coupled to a suction source (not shown) while the distal end is configured to be selectively placed in fluid communication with cutting edge 12 of the cutting instrument (see FIG. 1).

Apparatus 400 further comprises means for interrupting 425 and means for flushing 435. Means for interrupting 425 is disposed within handle 406 proximal to means for flushing 435, and preferably is disposed adjacent tubing section 22a, as depicted in FIG. 9. Means for interrupting 425 communicates with electrical supply means 413, and further communicates with actuation means 419.

Means for flushing 435 is disposed distal to means for interrupting 425, as depicted in FIG. 9, and also communicates with electrical supply means 413 and actuation means 419. Means for flushing 435 may fully encircle tubing section 22b, as depicted in FIG. 9, or may be disposed adjacent tubing section 22b.

In operation, a proximal end of electrical supply means 413 is coupled to a power source (not shown) and the proximal end of tubing 22 is coupled to a suction source (not shown). The apparatus may be provided in an “open” or fully aspirating state, permitting aspiration of fluid and cut tissue in a proximal direction through tubing 22. In the fully aspirating state, neither means for interrupting 425 nor means for flushing 435 applies substantial compressive forces upon tubing 22.

Once the cutting instrument becomes clogged, a physician may actuate means for interrupting 425, e.g., via actuation means 419. Like the embodiments described hereinabove, means for interrupting 425 is configured to apply compression upon tubing section 22a when actuated, thereby interrupting aspiration flow in tubing 22 (distal to section 22a).

In a next step, means for flushing 435 is actuated, e.g., via actuation means 419. Also like the embodiments described hereinabove, means for flushing 435 is actuated to apply compressive forces upon tubing section 22b, thereby flushing fluid in a distal direction. Distally urged fluid flushes clogged tissue away from cutting edge 12 of the cutting instrument.

Advantageously, in the embodiment of FIG. 9, means for interrupting 425 and means for flushing 435 may comprise various mechanically or electrically actuated means to apply compression to tubing sections 22a and 22b, respectively. For example, means for interrupting 425 may comprise any valve, solenoid, or other mechanism known in the art that may apply a compressive force upon tubing section 22a to compress the tubing in a manner as described hereinabove.

Similarly, means for interrupting 435 may comprise any valve, solenoid or pneumatic pump configured to apply a compressive force upon tubing section 22b. In a particularly useful embodiment, means for flushing 435 is configured to apply a circumferential compressive force upon tubing section 22b in a proximal to distal direction.

As will be apparent to one skilled in the art, a microprocessor and memory (not shown) may be employed to facilitate actuation of means for interrupting 425 and/or means for flushing 435. In response to data instructions received from the microprocessor, means for interrupting 425 and means for flushing 435 may be actuated accordingly.

In a particularly useful embodiment, the microprocessor is programmed to cause means for interrupting 425 to be actuated when actuation means 419, e.g., a button, is pressed once. The microprocessor then automatically instructs means for flushing 435 to be actuated at a later time, e.g., one second later. Such programming is particularly useful because it eliminates the need for a physician to actuate button 419 multiple times to achieve one cycle of flushing.

Alternatively, as will be apparent to one skilled in the art, actuation means 419 may comprise a multi-position switch configured to mechanically actuate means for interrupting 425 and means for flushing 435 in sequence. For example, when actuation means 419 comprises a button, and the button is partially depressed, means for interrupting 425 may be actuated. Then, when the button is fully depressed, means for flushing 435 subsequently is actuated.

Although one actuation means 419 has been described for actuating both means for interrupting 425 and means for flushing 435, other designs may be employed. For example, actuation means 419 may be configured to actuate means for interrupting 425 only, while a second actuation means (not shown) may be disposed on handle 406 to actuate means for flushing 435. In this embodiment, the microprocessor may have instructions to ensure that means for flushing 435 cannot be actuated when means for interrupting 425 is in an open position.

Further, as will be apparent to one skilled in the art, mechanical means similar to those described hereinabove with respect to FIGS. 2-8 may be employed to actuate means for interrupting 425 and/or means for flushing 435. For example, a bladder (not shown) may be disposed within handle 406 adjacent means for interrupting 425 and/or means for flushing 435. In this embodiment, a physician may compress one or more external regions of handle 406, which in turn compresses the bladder adjacent to means for interrupting 425 and/or means for flushing 435. This, in turn, causes compression of a corresponding section of tubing 22.

In an embodiment, means for interrupting and means for flushing may be positioned within a handle of the surgical cutting instrument. Actuation of these components may be accomplished electronically, e.g., by pressing a button disposed on the handle. In an embodiment, the components may be actuated mechanically.

In the device described in FIG. 9, means for interrupting 425 and/or means for flushing 435 may comprise one or more rollers that are configured to compress tubing 22, as described generally hereinabove with respect to FIGS. 5-8. In this embodiment, when actuation means 419 is actuated, a microprocessor (not shown) may provide instructions to one or more components, such as linkages, to cause the roller or rollers to compress tubing 22 as desired locations.

In yet a further alternative embodiment, actuation of means for interrupting 425 and means for flushing 435 may be achieved using a foot pedal (not shown). In this embodiment, the foot pedal may be coupled to a microprocessor that is programmed to actuate means for interrupting 425 and/or means for flushing 435, for example, when the foot pedal is depressed one or more times. Referring now to FIG. 10, a further alternative embodiment of the present invention is described. In FIG. 10, apparatus 400′ is similar to apparatus 400 of FIG. 9, with pertinent exceptions noted hereinbelow. Apparatus 400′ comprises irrigation supply line 439 having proximal and distal ends. The proximal end of the irrigation supply line 439 is coupled to an irrigation source (not shown) and the distal end is coupled to means for flushing 435′. In the embodiment of FIG. 10, means for flushing 435′ preferably comprises a one-way valve electronically coupled to actuation means 419, although other mechanical or electrical valves may be employed.

In operation, when an obstruction is detected in cutting edge 12, means for interrupting 425′ is actuated to interrupt aspiration by compressing tubing section 22b′, for example, using any of the techniques described hereinabove. In a next step, means for flushing 435′ is actuated. Specifically, in a preferred embodiment, an electronic signal is sent to means for interrupting 435′ to cause a one-way valve to open. The opening of the one-way valve permits fluid in irrigation line 439 to be advanced through the valve and towards tubing section 22c′. Irrigation fluid introduced through tubing section 22c′ then imposes pressure upon cutting edge 12 to flush tissue obstructions away from the cutting instrument.

As will be apparent to one skilled in the art, the pressure of the irrigation fluid introduced into tubing 22c′ may be adjusted to facilitate removal of the obstruction. Further, means for interrupting 425′ and means for flushing 435′ may be actuated in a pre-programmed sequence using a microprocessor, as described hereinabove with respect to FIG. 9, or may be actuated in sequence mechanically using a multi-position switch, as described hereinabove.

It should be noted that, in the embodiment of FIG. 10, three tubing sections 22a′-22c′ are employed. Tubing section 22a′ is sealingly coupled to tubing section 22b′, while tubing section 22b′ is sealingly coupled to tubing section 22c′. In this embodiment, tubing section 22b′ may comprises a more flexible material than sections 22a′ and 22c′ to facilitate compression of section 22b′.

It will be apparent to one skilled in the art that illustrative components of apparatus 400 may be interchanged with components of apparatus 400′, and vice versa. For example, apparatus 400 of FIG. 9 may comprise three separate tubing sections, as depicted in FIG. 10. Further, means for interrupting 425 of FIG. 9 may be disposed circumferentially about aspiration tubing, as depicted in FIG. 10, and so forth.

It will also be apparent to one skilled in the art that tissue obstructions may be detected by the physician, who then actuates the apparatus described hereinabove, or alternatively, the tissue obstructions may be detected using one or more sensors coupled to the cutting instrument. In the latter case, the sensors may detect a tissue obstruction directly, or may determine that an obstruction is present based on a reduction in flow likely to be associated with an obstruction. Once an obstruction is suspected, the sensors may relay a signal to automatically actuate the means for interrupting and/or means for flushing to facilitate removal of the obstruction with little or no physician intervention.

In an embodiment, the apparatus may include a disposable shaver blade set configured to be used with a conventional mechanical shaver handle. The disposable shaver blade set may include an inner cutting member and an outer sheath member. The inner cutting member may include an elongated shaft having a lumen disposed therein. The outer sheath member form a lumen disposed therein, which is configured to receive the elongated shaft of the inner cutting member. A disposable shaver blade set may be coupled to a distal region of the mechanical shaver handle such that a motor of the mechanical shaver handle drives the inner cutting member to debride bodily tissue.

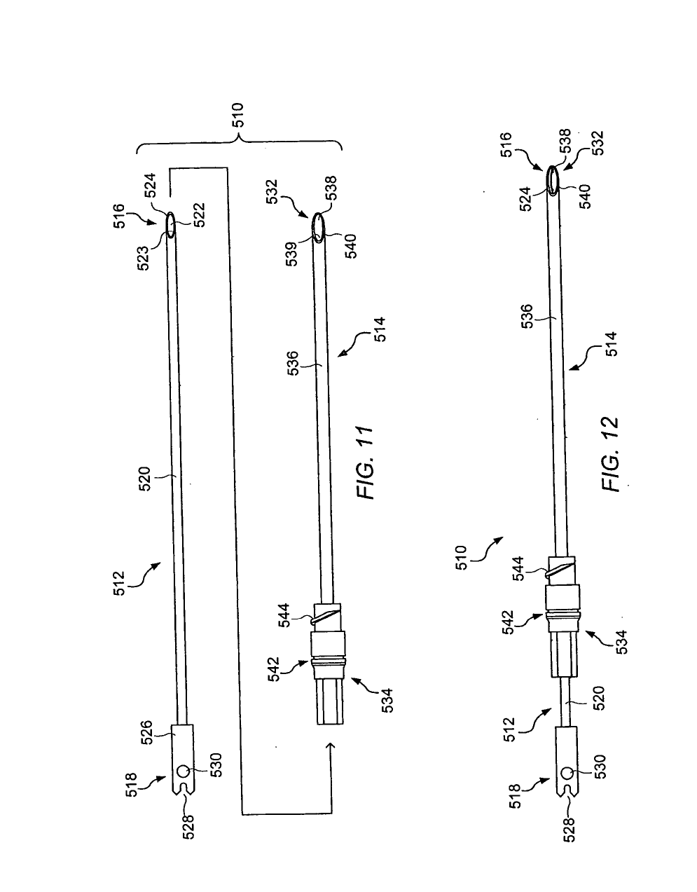

Referring now to FIG. 11, two components of a disposable shaver blade set 510 are described. The disposable shaver blade set 510 has an inner cutting member 512 and an outer sheath member 514. Inner cutting member 512 has a proximal end 518, a distal end 516 and an elongated hollow shaft 520 extending therebetween. The distal end 516 has a distal opening 522 and a cutting edge 524, while the proximal end 518 has proximal body 526, a shaped clutch 528 and a proximal opening 530.

The shaped clutch 528 is configured to be coupled to a motor of a mechanical shaver handle (not shown). The motor drives the inner cutting member to permit tissue to be debrided, as described in greater detail hereinbelow.

Proximal opening 530 of inner cutting member 512 is in fluid communication with the inside of elongated hollow shaft 520 and distal opening 522. During normal operation, fluid typically is suctioned into distal opening 522, then through lumen 523 in elongated shaft 520, through proximal opening 530, and through a lumen in the mechanical shaver handle.

Outer sheath member 514 has a proximal end 534, a distal end 532, and a hollow shaft 536 extending therebetween. The inner cutting member 512 is configured to be inserted into lumen 539 of outer shaft member 514, as indicated by the arrow of FIG. 11. The assembled components are depicted in FIG. 12.

The proximal end 534 of outer sheath member 514 has an anchoring means 542, which is configured to attach outer sheath member 514 to the mechanical shaver handle. The proximal end 534 has an optional attachment means 544 for attaching the outer sheath member 514 to an access cannula (not shown).

In an embodiment, rotation of the inner cutting member during use may be driven via the mechanical shaver handle. Debrided tissue is aspirated through the lumen of the inner cutting member. When an obstruction is present in the disposable shaver set, a means for interrupting may be actuated so as to interrupt suction at the distal cutting edge of the inner cutting member. The distal end 532 of outer sheath member 514 has a distal opening 538 and a part-off edge 540, as shown in FIG. 11. Part-off edge 540 is configured to sever tissue drawn into the distal opening 538 when the cutting edge 524 of the inner cutting member 512 is rotating within the distal opening. In particular, a distal region 516 of the inner cutting member 512 is seated against distal region 532 of the outer sheath member 514, such that the cutting edge 524 is aligned for opposition to the part-off edge 540.

A common problem associated with the use of surgical cutting instruments during arthroscopic procedures is the tendency of cut tissue to become clogged in the vicinity of distal opening 538 and cutting edge 524 of the instrument. In accordance with one aspect of the present invention, apparatus and methods described hereinbelow are provided to quickly and efficiently expel clogged tissue from distal opening 538 without the need to remove shaver blade set 510 from the mechanical shaver handle or the operative site.

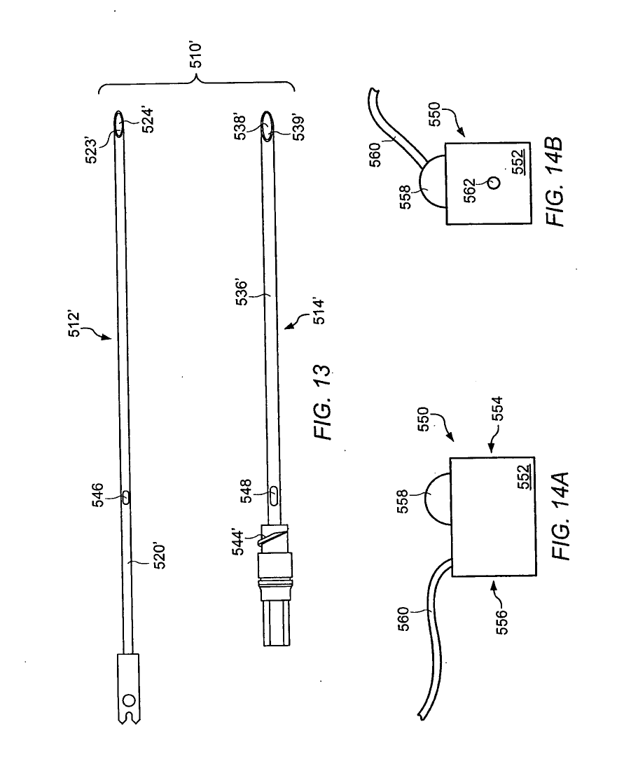

In an embodiment, both the inner cutting member and the outer sheath member may include at least one side port. The side ports of the inner cutting member and the outer sheath member may at least partially overlap. A fluid supply line coupled to an irrigation source, may be provided in fluid communication with the side ports of the inner cutting member and outer sheath member. Referring now to FIGS. 13-15, another embodiment of the present invention is described wherein the same or similar reference numbers refer to the same or similar structure. In FIG. 13, an inner cutting member 512′ is similar to inner cutting member 512 of FIGS. 11-12, with a main difference being that a side port 546 is disposed in a lateral surface of the elongated shaft 520′. Similarly, outer sheath member 514′ has been modified by adding side port 548 along a lateral surface of shaft 536′. It should be noted that both side ports 546 and 548 are in fluid communication with lumens 523′ and 539′ of inner cutting member 512′ and outer sheath member 514′, respectively.

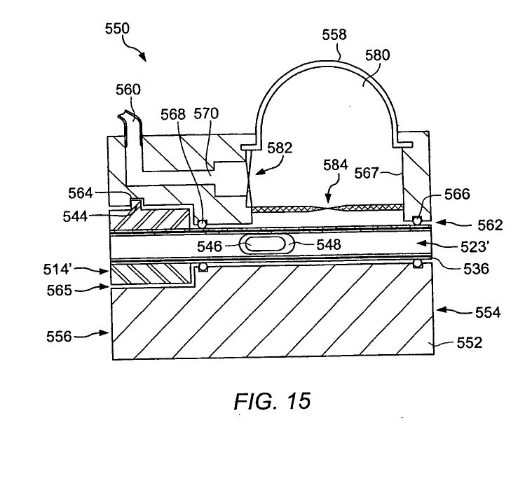

Referring now to FIG. 14A, apparatus 550 is configured for use with disposable shaver blade set 510′ of FIG. 13 to facilitate removal of obstructions from the distal opening or cutting edge during a surgical procedure. Apparatus 550 comprises a means for flushing fluid in a distal direction, as described in greater detail with respect to FIG. 15 below.

In FIG. 14A, a side view of apparatus 550 is shown. Apparatus 550 includes a housing 552 having a proximal end 556, a distal end 554, a fluid supply line 560 and an actuation means 558. In FIG. 14B, a front view of apparatus 550 reveals that the apparatus has a through port 562 disposed in a lateral surface of a distal wall 554. Through port 562 forms a conduit that extends proximally through the housing 552. Further, a through port 562 is in fluid communication with a bore 565 formed at the proximal end 556 as shown in FIG. 15 hereinbelow.

Referring now to FIG. 15, a first application of the present invention is described. In FIG. 15, disposable shaver set 510′ of FIG. 13 is shown used in conjunction with apparatus 550 of FIGS. 14A-14B.

Housing 552 of apparatus 550 has a securing attachment 564, which is disposed near the proximal end 556 of the housing. As shown in FIG. 15, the securing attachment 564 is configured to be coupled to the attachment means 544′ of the outer sheath member 514′. The securing attachment 564 and the attachment means 544′ may be coupled together in any suitable manner, for example, using a snap-lock engagement or by rotatingly threading attachment means 544′ into engagement with means for securing 564. Once these components are coupled together, longitudinal movement of the outer sheath member 514′ with respect to housing 552 is substantially prohibited.

The housing 552 also includes a proximal sealing ring 568 and a distal sealing rings 566. The proximal and distal sealing rings 568, 566 are configured to provide a fluid tight seal around the exterior surface of the shaft 536′ of the outer sheath member 514′. Proximal and distal sealing rings 568 and 566 prohibit fluid movement within the port 562 for purposes described hereinafter.

Referring still to FIG. 15, the housing 552 also has a reservoir 580. The reservoir 580 is surrounded by an actuation means 558, a first one-way valve 582, a second one-way valve 584, and a wall 567 of the housing 552, as depicted in FIG. 15.

The actuation means 558 is attached to the housing 552 in such a manner that it forms a bulb-shaped member, as depicted in FIG. 15. Actuation means 558 may be manufactured using a suitable biocompatible compound that allows the actuation means to be compressed in an inward direction when a force is applied, and then return to its original, non-compressed state (shown in FIG. 15) when the compressive force is removed.

Irrigation fluid from the fluid supply line may be distally injected into the lumen of the inner cutting member via the side ports of the inner cutting member and outer sheath member. The irrigation fluid may be injected into the side ports at a location distal to the means for interrupting. The irrigation fluid introduced into the lumen of the inner cutting member flows distally towards the end of the disposable shaver set, clearing the obstructing material from the cutting edge of the surgical instrument. In order to facilitate the introduction of irrigation fluid into the side ports of the inner cutting member and outer sheath member, a suitable housing may be employed. The housing preferably includes a fluid reservoir configured to temporarily check fluid flow from the supply line. Preferably, a first one-way valve is provided to permit fluid from the supply line to enter the reservoir of the housing. A second one-way valve is provided that contains the fluid in the reservoir until an actuation means is actuated, thereby urging fluid distally into the lumen of the inner cutting member. The apparatus 550 also includes a delivery channel 570 having proximal and distal regions. Fluid supply line 560 is coupled to the proximal region of a delivery channel 570, as shown in FIG. 15. The first one-way valve 582 separates the distal region of delivery channel 570 from reservoir 580 and the second one-way valve 584 separates reservoir 580 from through port 562 of housing 50, as shown in FIG. 15

In an embodiment of a method of operation, the fluid supply line 560 provides irrigation fluid to the delivery channel 570. The first one-way valve 582 permits fluid flow in a distal direction, i.e., from delivery channel 570 into the reservoir 580. Irrigation fluid is collected in the reservoir 580, and cannot flow in a proximal direction back into the delivery channel 570.

When actuation means 558 is compressed, e.g., manually, irrigation fluid in the reservoir 580 is urged in a distal direction through the second one-way valve 584. Irrigation fluid flows distally through the second one-way valve 584 into through port 562, but cannot flow in a proximal direction back into the reservoir 580. Irrigation fluid is injected into disposable shaver set 510′ via side ports 548 and 546 of the outer sheath member 514′ and inner cutting member 512′, respectively. As shown in FIG. 15, the side ports 548 and 546 at least partially overlap to permit the introduction of irrigation fluid into lumen 523′ of inner cutting member 512′ of FIG. 13.

It should be noted that irrigation fluid flowing distally through the second one-way valve 584 is urged into lumen 523′ in part because the fluid cannot flow proximally past proximal sealing ring 568 and cannot flow distally beyond distal sealing ring 566. Therefore, fluid flowing from the reservoir 570 into through port 562 of the housing 552 is urged into lumen 523′ via side ports 546 and 548.

In accordance with one aspect of the present invention, irrigation fluid that is channeled into lumen 523′ is urged in a distal direction, i.e., towards the opening 538′ and the cutting edge 524′, because a means for interrupting (not shown in FIGS. 13-15) is previously actuated. The means for interrupting inhibits aspiration flow in lumen 523′ at a location proximal to the side ports 546 and 548.

Specifically, during operation of disposable shaver set 510′, when a physician suspects that an obstruction is present in the vicinity of distal opening 538′ and/or cutting edge 524′, the means for interrupting is actuated. The means for interrupting interrupts the provision of aspiration to distal opening 538′ and cutting edge 524′ by blocking aspiration flow through lumen 523′ at a location proximal to the means for flushing.

The means for interrupting is not shown in FIGS. 13-15 for illustrative purposes only. However, several different means for interrupting may be employed to inhibit the provision of aspiration within lumen 523′ such as those described herein and in U.S. patent application Ser. No. 10/782,489, filed February 18 which is incorporated herein by reference in its entirety. The means for interrupting may be disposed in the mechanical shaver handle itself, or coupled to aspiration tubing extending between the suction source and the handle.

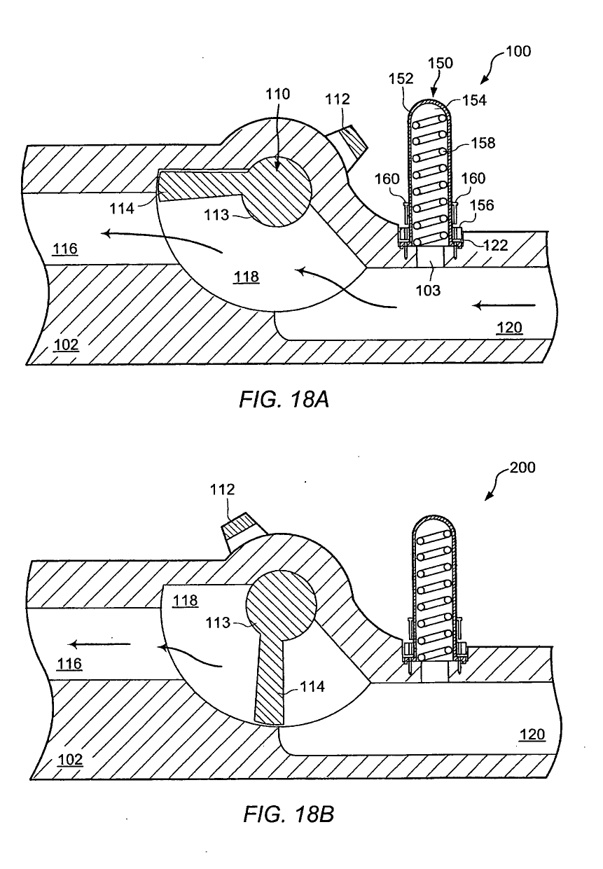

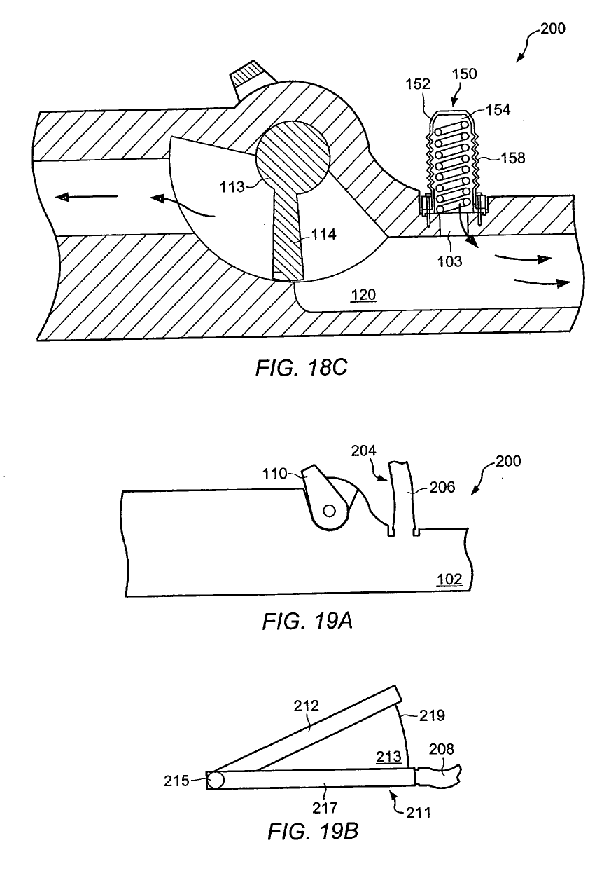

Alternatively, a means for interrupting that may be used in the embodiment of FIGS. 13-15 is described fully with respect to FIGS. 18-22 hereinbelow. Specifically, means for interrupting of FIGS. 8-12 may be employed to inhibit aspiration flow within the mechanical shaver handle.

Further, it will be apparent to one skilled in the art that means for interrupting also may be disposed in, or coupled to, the apparatus 550 of FIGS. 14-15. In keeping with the spirit of the present invention, any number of means for interrupting may be employed to interrupt the provision of aspiration through lumen 523′, the means for interrupting being disposed proximal to the means for flushing.

Referring still to FIG. 15, and with the means for interrupting actuated, the actuation of actuation means 558 applies pressure to irrigation fluid in reservoir 580, thereby causing fluid from the reservoir 580 to flow distally in lumen 523′. The distally flowing fluid flushes clogged tissue away from distal opening 538′ and the cutting edge 524′ of the surgical instrument. Once the compressive force imposed upon actuation means 558 is released, aspiration flow may be restored throughout lumen 523′. The previously-clogged tissue then may be introduced back into cutting edge 524′, reduced in size, and then effectively aspirated through lumen 523′.

It should be noted that once the compressive force imposed on actuation means 558 is removed, fluid from supply line 560 passes through the first one-way valve 582 and replenishes the fluid supply in the reservoir 580. In this manner, actuation means 558 may be repeatably depressed, as needed, to provide multiple flushes in order to improve the likelihood that clogged tissue is removed.

Advantageously, obstructing tissue may be removed from the disposable shaver set using actuation means 558 without having to remove the cutting instrument from the surgical site. Also, the surgeon is not expected to have to replace the disposable shaver set with a different instrument, thereby saving time and money.

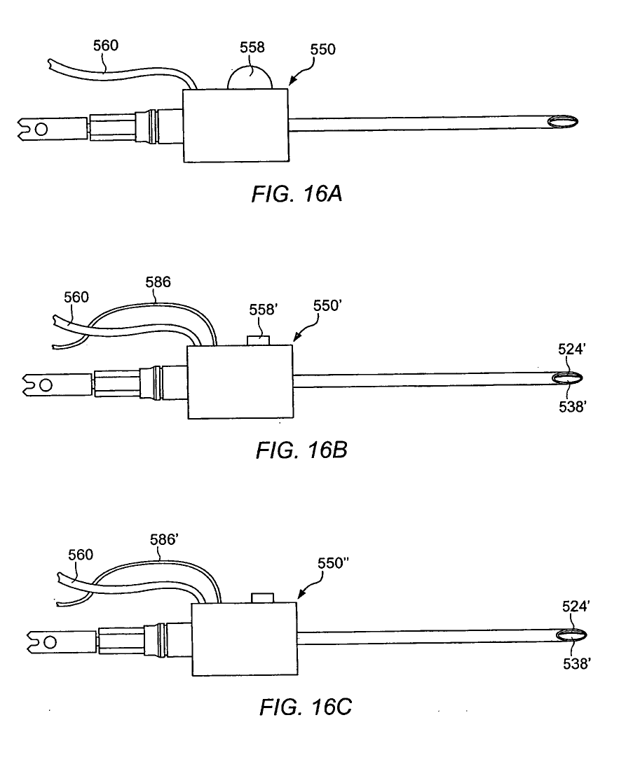

Referring now to FIGS. 16A-16C, various actuation means for actuating the means for flushing of FIGS. 14-15 are described. In FIG. 16A, actuation means 558 is the same as the actuation means described in FIGS. 14-15, and is provided for illustrative purposes only.

In FIG. 16B, alternative actuation means 558′ is coupled to housing 550′, and further coupled to an electrical supply line 586. Preferably, the embodiment of FIG. 16B comprises similar sealing rings, one-way valves and means for securing, as was described in detail in FIG. 15 and those features are expressly incorporated here. However, in the embodiment of FIG. 16B, an electro-mechanical actuator (not shown), such as a piston, is used to inject fluid from supply line 560 into lumen 538′ and towards the cutting edge 524′. Specifically, depressing actuation means 558′ activates the electro-mechanical actuator, which is powered by electrical supply line 586.

In the embodiment of FIG. 16C, the actuation means employed (not shown) is similar to FIG. 16B, but the electro-mechanical actuator of FIG. 16B may be activated using a foot pedal, an actuator mounted on the mechanical shaver handle, or an actuator mounted outside the sterile field. Such actuation means are well known in the art and are often used to trigger powered equipment in the operative field.

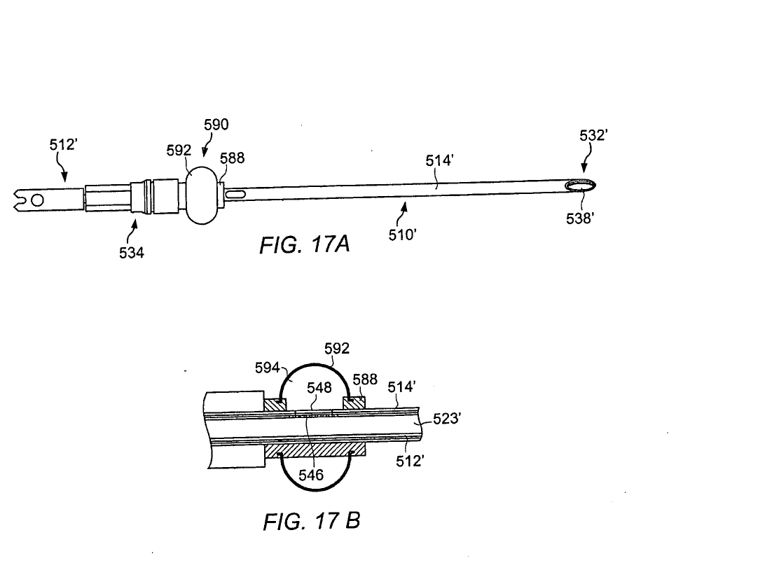

Referring now to FIGS. 17A-17B, an alternative means for flushing 590 is described for clearing obstructions from surgical cutting instruments. In FIG. 17A, means for flushing 590 is integrated into the outer sheath member 514′ of disposable shaver blade set 510′.

In the embodiment of FIGS. 17A-17B, inner cutting member 512′ and outer sheath member 514′ of FIGS. 13-14 are employed. Inner cutting member 512′ comprises elongated shaft 520′ and lumen 523′ disposed therein. Further, as described above, side port 546 is formed in a lateral surface of inner cutting member 512′, as shown in FIG. 17B. Similarly, outer sheath member 514′ comprises an elongated shaft having side port 548 disposed therein, and lumen 539′ for accommodating inner cutting member 512′.

In an alternative embodiment, the means for flushing includes a reservoir coupled directly to an exterior surface of the disposable shaver set. In this embodiment, mounting means are provided on the outer sheath member, and a bulb-shaped member is coupled to the mounting means. A fluid reservoir is formed between the bulb-shaped member and the outer sheath member. In FIGS. 17A-17B, means for flushing 590 has actuation means 592, which preferably is attached to mounting means 588 in such a manner that it forms a bulb-shaped member, as depicted in FIG. 17B. Actuation means 592 and mounting means 588 enclose a fluid reservoir 594.

Actuation means 592 may be manufactured using a suitable biocompatible compound that allows the actuation means to be compressed in an inward direction when a force is applied, and then return to its original, non-compressed state (depicted in FIG. 17B) when the compressive force is removed.

When an obstruction is detected, a means for interrupting, which is located proximal to the fluid reservoir, is actuated to interrupt the provision of suction to the distal cutting edge of the inner cutting member. Then, in a next step, the means for flushing is actuated by depressing the bulb-shaped member to cause fluid from the reservoir to be injected into the side ports of the inner cutting member and outer sheath member. Injected fluid then flows distally within the lumen of the inner cutting member towards the cutting edge to facilitate removal of clogged tissue. In an embodiment, a method of operation when an obstruction is detected in the cutting instrument, may include actuating the means for interrupting (not shown in FIGS. 17A-17B). As described below with respect to FIGS. 18-22, the means for interrupting is disposed proximal to the means for flushing, and inhibits aspiration flow to a distal section of lumen 523′.

After the means for interrupting is actuated, means for flushing 590 may be actuated. Specifically, compression of actuation means 592 forces fluid contained in fluid reservoir 594 into lumen 523′ via side ports 548 and 546. Fluid injected into lumen 523′ is urged in a distal direction to clear clogged tissue from opening 538′. Of course, fluid is urged in a distal direction because the means for interrupting prevents irrigation fluid from flowing proximally towards the suction source.

Advantageously, the fluid reservoir may be refilled simply by relieving the compressive force applied to the bulb-shaped member. Specifically, a vacuum force causes fluid in the lumen to return to the fluid reservoir, via the side ports, once compression is removed. This allows repeated compression of the bulb-shaped member to repeatedly flush the cutting edge of the instrument. When compression of actuation means 592 is relieved, then a vacuum force causes fluid from lumen 523′ to be brought back into fluid reservoir 594 via side ports 546 and 548. In this manner, a physician may repeatedly compress and release actuation means 592 to repeatedly flush particulate matter from distal opening 538′.

It will be apparent to one skilled in the art that, in the embodiments of FIGS. 13-15 and FIGS. 17A-17B, each side port 546 and 548 may comprise multiple openings or one single opening. Further, the configurations of the openings may be varied to achieve the objectives of the present invention.

Referring now to FIGS. 18A-18C, a further alternative embodiment of the present invention is described. In this embodiment, apparatus 100 comprises mechanical shaver handle 102, means for interrupting 110 and means for flushing 150.

Mechanical shaver handle 102 preferably is similar to a conventional handle used in surgical cutting instruments, except as noted below. A conventional disposable shaver set, such as disposable shaver set 510 of FIGS. 11-12, may be coupled to a distal region of handle 102 to debride tissue, as generally set forth above. As will be apparent to one skilled in the art, a motor (not shown) is incorporated into handle 102 to drive inner cutting member 512 of FIG. 11. Further, a suction source (not shown) is coupled to a proximal region of handle 102, such that the suction source is in fluid communication with proximal lumen section 116 of handle 102, as described in further detail hereinbelow.

In any of the embodiments described herein, the means for interrupting may include a rotating valve member coupled to the mechanical shaver handle. In operation, rotation of an actuation means coupled to the rotating valve member causes the valve member to block aspiration flow through the lumen of the mechanical shaver handle. With fluid flow interrupted, the means for flushing injects fluid into the lumen at a location distal to the means for interrupting, thereby causing injected fluid to flow distally towards the distal cutting edge of the instrument.

In the embodiment of FIGS. 18A-18C, means for interrupting 110 comprises rotating valve member 114, rotational joint 113 and actuation means 112. Joint 113 is coupled to an interior section of mechanical shaver handle 102 such that actuation means 112 and rotating valve member 114 can rotate about a pivot point. For example, rotational joint 113 may comprise a laterally-extending shaft disposed through a complementary bore (not shown) formed in an upper surface of handle 102. The laterally-extending shaft is configured for circumferential rotation within the bore, thereby permitting rotational movement of rotating valve member 114 and actuation means 112.

As will be shown in greater detail below, rotating valve member 114 is disposed for rotation within fluid cut-off chamber 118, which is situated between proximal lumen section 116 and distal lumen section 118 of handle 102.

Referring still to FIGS. 18A-18C, means for flushing 150 comprises actuation means 152, fluid reservoir 154 and means for refilling 158. Actuation means 152 may comprise any suitable compliant biocompatible membrane that may be deformed as shown in FIG. 18C below. In a preferred embodiment, means for refilling 158 comprises a spring disposed within the biocompatible membrane of actuation means 152. The spring is configured to return actuation means 152 to the position depicted in FIG. 18A, as described in further detail below.

Means for flushing 150 preferably further comprises anchor bracket 156 and means for securing 160. Mechanical shaver handle 102 preferably comprises anchor pocket 122 disposed in a lateral surface of the handle, which is configured to receive anchor bracket 156, as depicted in FIG. 18A. In one embodiment, means for securing 160 comprises at least two bolts 160 that are configured to be received by bores formed in anchor bracket 156 and handle 102. Accordingly, the bolts secure means for flushing 150 to handle 102, as shown in FIG. 18C.

It should be noted that in FIGS. 18A-18B, anchor bracket 156 and means for securing 160 are not secured to mechanical shaver handle 102, while in FIG. 18C, the components are secured together. This is for illustrative purposes only. During normal operation, described below, means for flushing 150 would be secured to handle 102 as shown in FIG. 18C. Further, as will be apparent to one skilled in the art, numerous other means for securing may be employed to secure means for flushing 150 to handle 102.

Mechanical shaver handle 102 further comprises at least one side port 103, which is formed in a lateral surface of the handle. Side port 103 is disposed for fluid communication with reservoir 154 of means for flushing 150, as shown in FIGS. 18A-18C.

A preferred method of using apparatus 100 of FIGS. 18A-18C will now be described. In a first step, shown in FIG. 18A, rotating valve member 114 is provided in an open position. When suction is applied by the suction source coupled to the proximal end of the handle, aspiration is provided throughout proximal and distal lumen sections 116 and 120, thereby providing aspiration to cutting edge 524 and distal opening 538 of disposable shaver set 510 to remove cut tissue.

If a physician detects that cut tissue has clogged distal opening 538 or cutting edge 524, the physician first rotates means for actuating 112 in a counter-clockwise direction. This causes rotating valve member 114 to be advanced counter-clockwise via rotational joint 113. Assuming a sufficient degree of rotation is achieved, then rotating valve member 114 is transformed to a closed position, as depicted in FIG. 18B. In the closed position, valve member 114 prohibits aspiration of fluid distal to the valve member. It should be noted that, at this time, aspiration may still occur in proximal lumen section 116 because the suction source need not be turned off each time an obstruction is detected. Rather, the actuation of means for interrupting 110 blocks the flow of aspiration through distal lumen section 120 to the surgical site.

In a next step, depicted in FIG. 18C, means for flushing 150 is actuated. Actuation may be achieved by compressing actuation means 152 to force fluid contained in reservoir 154 in a distal direction through side port 103, as indicated by the arrows in FIG. 18C. Preferably, the complaint membrane of actuation means 152 substantially conforms to the compressive forces applied.

Fluid that is injected distally through side port 103 flows distally through distal lumen section 120. It should be noted that the injected fluid cannot flow proximally since rotating valve member 114 blocks flow through chamber 118. In accordance with one aspect of the present invention, fluid injected into distal lumen section 120 flows distally towards the cutting edge of the surgical instrument to facilitate removal of clogged tissue.

Once flushing occurs, a physician may relieve the compressive force imposed upon actuation means 152. Means for refilling 158, such as a spring, causes the membrane of actuation means 152 to return to the position depicted in FIG. 18B. When actuation means 152 returns to its expanded position of FIG. 18B, a vacuum force causes reservoir 154 to be refilled with fluid from distal lumen section 120. With reservoir 154 refilled, a physician may subsequently compress actuation means 152 to repeat flushing of the cutting edge, and the process may be repeated as necessary until clogged tissue is expelled.

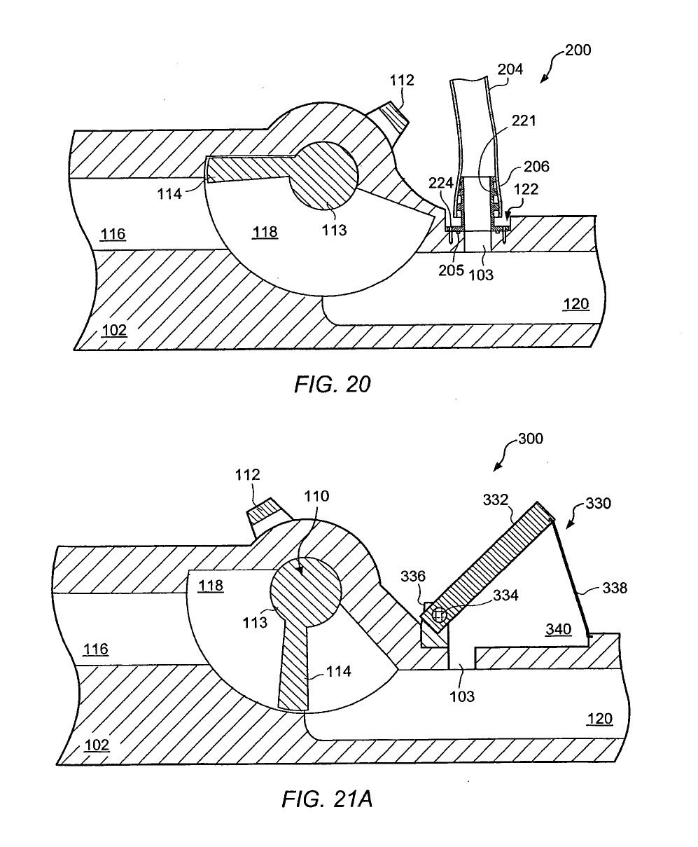

Referring now to FIGS. 19-20, a further alternative embodiment of the present invention is described. In this embodiment, apparatus 200 comprises mechanical shaver handle 102, means for interrupting 110, fluid supply means 204 and means for flushing 211. Mechanical shaver handle 102 and means for interrupting 110 preferably are provided as described in FIGS. 18A-18C hereinabove, except as noted below.

In FIGS. 19-20, apparatus 200 comprises fluid supply line 204 having proximal end 208 and distal end 206. Proximal end 208 is coupled to means for flushing 211, as shown in FIG. 19B.

Means for flushing 211 is in the general form of a foot pedal and comprises base plate 217, deflection plate 212, fluid reservoir 213 and membrane 219, which is coupled between deflection plate 212 and base plate 217. Base plate 217 is coupled to deflection plate 212 at pivot point 215. As will be apparent to one skilled in the art, compression of deflection plate 212 in a direction towards base plate 217 forces fluid contained in reservoir 213 through fluid supply line 204 and into distal lumen section 120 of handle 102.

Referring now to FIG. 20, distal end 206 of fluid supply line 204 is disposed for fluid communication with side port 103 of mechanical shaver handle 102. Distal end 206 of fluid supply line 204 preferably is secured to fluid supply coupler 221, as depicted in FIG. 20. Further, in a preferred embodiment, mechanical shaver handle 102 comprises anchor pocket 122, which is configured to receive a distal section of fluid supply coupler 221. Fluid supply coupler 221 preferably is secured to mechanical shaver handle 102 using attachment screws 224, and this connection is sealed against fluid leaks by sealing ring 205.

The operation of apparatus 200 is similar to the operation of apparatus 100 of FIGS. 18A-18C, except as noted below. During normal operation, means for interrupting 110 is provided in the open position depicted in FIG. 20 to permit aspiration throughout proximal and distal lumen sections 116 and 120. When a possible tissue obstruction is detected in the vicinity of cutting edge 24, then actuation means 112 of means for interrupting 110 is actuated to cause rotating valve member 114 to block chamber 118, as described in FIG. 18B above.

In a next step, a physician may actuate means for flushing 211, e.g., by stepping on deflection plate 212 to compress fluid contained in reservoir 213. The fluid contained in reservoir 113 then flows through fluid supply line 204 and into distal lumen section 120 via side port 103 of handle 102. When rotating valve member 114 is in a closed position, as described in FIG. 18C hereinabove, then fluid injected into distal lumen section 120 is urged in a distal direction towards distal opening 538. In this manner, fluid flushes clogged tissue away from cutting edge 524 and distal opening 538 of the cutting instrument. Further, as described hereinabove, means for flushing 211 may be repeatedly actuated, e.g., by compressing and releasing deflection plate 212, to repeatedly flush the cutting edge and distal opening, thereby facilitating removal of clogged tissue.

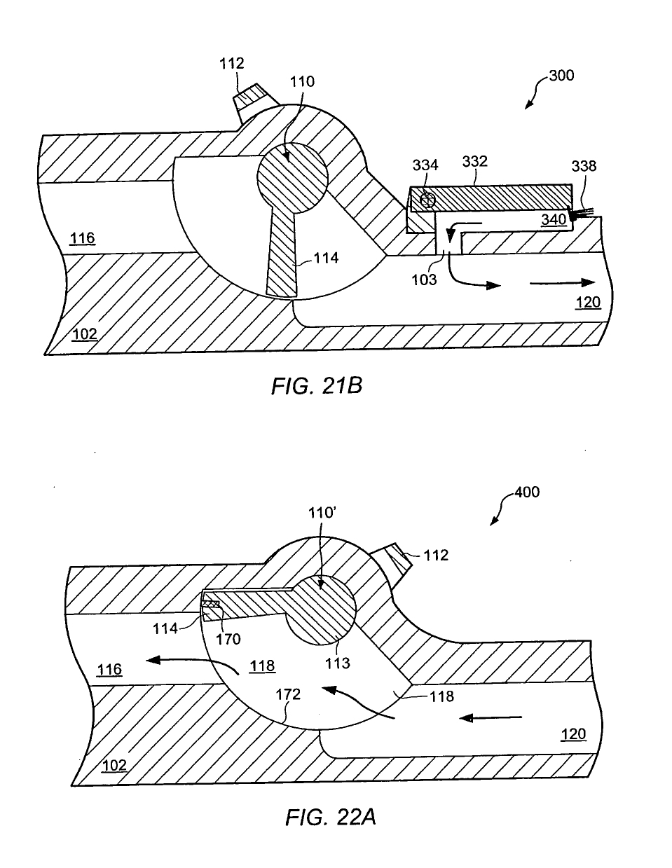

Referring now to FIGS. 21A-21B, a further alternative embodiment of the present invention is described. In this embodiment, apparatus 300 comprises mechanical shaver handle 102, means for interrupting 110 and means for flushing 330. Mechanical shaver handle 102 and means for interrupting 110 preferably are provided as described in FIGS. 18A-18C hereinabove, except as noted below.

In FIGS. 21A-21B, means for flushing 330 comprises actuation means 332, membrane 338, and fluid reservoir 340 contained therein. Membrane 338 preferably is coupled between actuation means 332 and a lateral surface of mechanical shaver handle 102, as depicted in FIG. 21A. Reservoir 340 is in fluid communication with distal lumen section 120 of mechanical shaver handle 102 via side port 103.

Means for flushing 330 further comprises means for securing 336 and pivot means 334. Means for securing 336 may be in the form of a block member that is attached to shaver handle 102, as shown in FIG. 21A. Pivot means 334 couples means for securing 336 to actuation means 332 and allows rotational movement of actuation means 332 with respect to handle 102.

The operation of apparatus 300 is similar to the operation of apparatus 100 of FIGS. 18A-18C, except as noted below. During normal operation, means for interrupting 110 is provided in the open position depicted in FIG. 18A to permit aspiration throughout proximal and distal lumen sections 116 and 120. When a physician detects a possible tissue obstruction near cutting edge 524, then actuation means 112 of means for interrupting 110 is actuated to cause rotating valve member 114 to block chamber 118, as depicted in FIG. 21A.

In a next step, a physician may actuate means for flushing 330, e.g., by manually compressing actuation means 332 towards handle 102. Actuation means 332 pivots about pivot means 334 and urges fluid in reservoir 340 through side port 102 and into distal lumen section 120, as depicted in FIG. 21B. During compression, membrane 338 collapses to permit actuation means 332 to be deflected as far as possible.

As described hereinabove, when rotating valve member 114 is in a closed position, fluid injected into distal lumen section 120 is urged in a distal direction towards distal opening 538. In this manner, fluid flushes clogged tissue away from cutting edge 524 and distal opening 538 of the cutting instrument.

Further, means for flushing 330 may be repeatedly actuated, e.g., by compressing and releasing actuation means 332. Each time actuation means 332 is released, the actuation means pivots about pivot means 334 and returns to the position depicted in FIG. 21A. During this time, reservoir 340 is refilled with fluid from distal lumen section 120. In this manner, a physician may repeatedly flush the cutting edge and distal opening, thereby facilitating removal of clogged tissue.

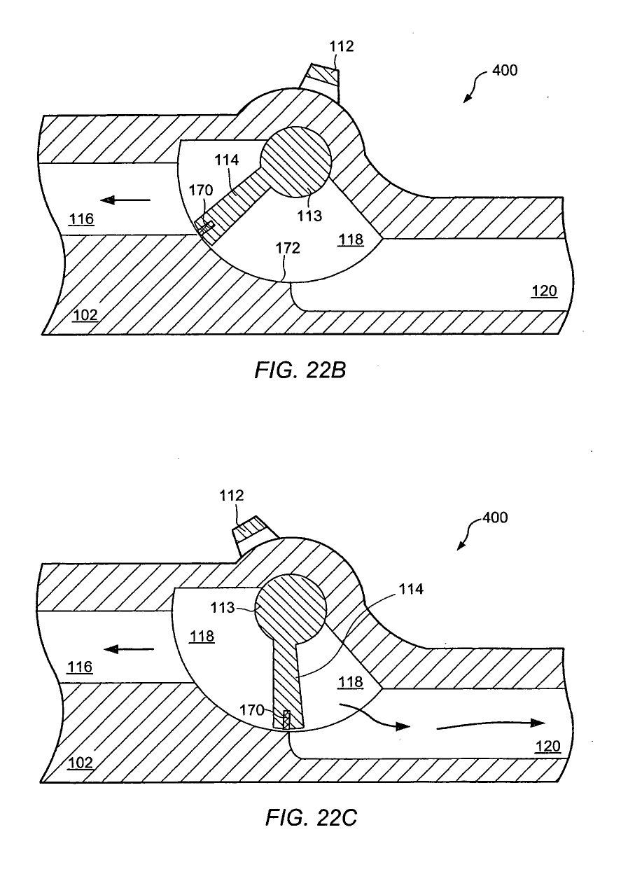

Referring now to FIGS. 22A-22C, yet a further alternative embodiment of the present invention is described. In this embodiment, apparatus 400 comprises mechanical shaver handle 102 and means for interrupting 110′. Means for interrupting 110′ also serves as a means for flushing, as will be described in greater detail hereinbelow.

Means for interrupting 110′ is similar to means for interrupting 110 of FIGS. 8A-8C, and comprises actuation means 112, rotational joint 113 and rotating valve member 114. In this embodiment, fluid seal 170 is coupled to an outer edge of rotating valve member 114, as depicted in FIG. 22A. Fluid seal 170 may comprise any suitable compliant biocompatible material that sealingly engages curved interior 172 of chamber 118 in handle 102, for purposes described hereinafter.

During normal operation, means for interrupting 110′ is provided in the open position depicted in FIG. 22A to permit aspiration throughout proximal and distal lumen sections 116 and 120. When a physician detects a possible tissue obstruction in the vicinity of cutting edge 24, then actuation means 112 is rotated counter-clockwise to cause rotating valve member 114 to block chamber 118, as shown in FIG. 22B. At this time, aspiration in distal lumen section 120 is halted.

In a next step, a physician may further rotate actuation means 112 counter-clockwise to cause valve member 114 to rotate counter-clockwise along curved interior 172 of handle 102, as depicted in FIG. 22C. As valve 114 is rotated counter-clockwise, fluid in distal lumen section 120 is flushed in a distal direction, i.e., towards distal opening 538 and cutting edge 524. In this manner, fluid flushes clogged tissue away from the cutting edge and distal opening of disposable shaver set 10.

If a physician needs to repeatedly flush the cutting edge of the instrument, then the physician may repeatedly rotate actuation means 112 clockwise and counter-clockwise to recreate the flushing effect provided by rotating valve member 114.

If desired, curved interior 172 may be provided with a distal stop (not shown) to inhibit further distal movement of rotating valve member 114. Such a distal stop would ensure that seal 170 does not relinquish its sealing engagement with curved interior 172, thereby ensuring that flushed fluid does not travel proximally back into proximal lumen section 116.

It will be apparent to one skilled in the art that certain illustrative components in one embodiment hereinabove may be interchanged with components of another embodiment to achieve the objectives of the present invention. For example, if a fluid reservoir is employed, the fluid reservoir may be any one of the designs described hereinabove with respect to FIG. 15 and FIGS. 17-21.

Further, as will be apparent to one skilled in the art, the shape and size of the reservoir may be varied to improve the ability to remove tissue obstruction from the cutting edge of the instrument. Similarly, any of the actuation means described hereinabove may be designed such that the rate of compression is varied to improve removal of obstructions from the cutting edge.

Also, in the embodiment of FIGS. 22A-22C, it will be apparent to one skilled in the art that the configurations of chamber 118, rotating valve member 114 and other components may be optimized to provide a sufficient volume and pressure of fluid that is flushed in a distal direction to facilitate removal of obstructions.

It will also be apparent to one skilled in the art that tissue obstructions may be detected by the physician, who then actuates the apparatus described hereinabove, or alternatively, the tissue obstructions may be detected using one or more sensors coupled to the cutting instrument. In the latter case, the sensors may detect a tissue obstruction directly, or may determine that an obstruction is present based on a reduction in flow likely to be associated with an obstruction. Once an obstruction is suspected, the sensors may relay a signal to automatically actuate the means for interrupting and/or means for flushing to facilitate removal of the obstruction with little or no physician intervention.

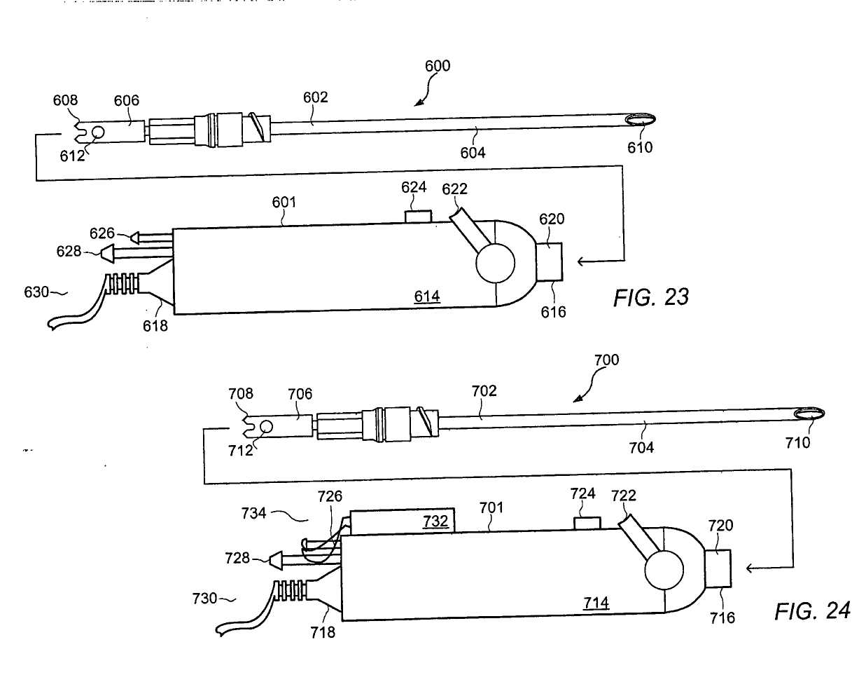

Now referring to FIG. 23 a different embodiment 600 of the present invention is shown comprised of a standard tubular shaver attachment 602 and a shaver power handle 601. In this view the disposable shaver attachment 602 is not yet connected to the handle, but would be by attaching the attachment's proximal end 608 to the handles distal end 616 as shown by the arrow.

The shaver attachment 602 is comprised of an outer tube 604 and an inner cutting tube 606 which is configured to slide into and rotate within the outer tube. Both the inner and outer tubes have distal openings 610 which are configured to draw tissue in and resect it. The proximal end 608 of the inner tube is configured with a suction port 612 which is in communication with the distal opening 610.

Now turning to the shaver handle 601, it is comprised of a handle body 614, and distal end 616 and a proximal end 618. The distal end contains a distal coupling 620 configured to receive and lock in the proximal end of the shaver attachment 608. The proximal end contains a power line 630, a suction lumen coupler 628 and an aspiration lumen coupler 626. The aspiration lumen coupler 626 is configured to accept a gas line (not shown) which is attached to a pressurized gas source as is typically found in the operating room. As well the suction lumen coupler 628 is configured to accept a suction line (not shown) which is attached to a suction source as is typically found in the operating room. Finally the power line 630 is connected to a shaver controller (not shown) as is typically used in the operating room. Also on the shaver handle body 614 are a suction flow control actuator 622 and an aspiration flow actuator 624.

When the shaver attachment 602 is attached to the shaver handle 601 and a suction source (not shown) to the suction lumen coupler 628, with the suction flow actuator 622 in the on position, fluid and tissue is drawn into the distal opening 610 through the inner tube 606, out of the proximal opening of the inner tube 612 and through the shaver handle to the suction lumen coupler 628. When tissue is clogged in the distal opening 610 and cannot be resected cleanly, the surgeon can move the suction flow actuator to the interrupt or off position stopping the flow of fluid into the distal end of the shaver attachment, as has been described above. Then the surgeon can activate the aspiration flow actuator 624, which opens a valve inside the shaver handle body 614 and connects the high pressure gas connected to the aspiration lumen coupler 626. This pressurized gas forces the clogged tissue distally out of the shaver attachment without the need for the surgeon to remove the shaver from the operating sight. The aspiration flow actuator can be configured to allow a continuous flow of gas through the distal opening 610 while it is activated (by depressing it for example) or a predetermined short burst, which is repeated each time the actuator is activated. In one embodiment the aspiration flow actuator 624 is configured such that it can only be activated when the suction flow actuator 622 is in the off or flow interrupted position.

Now turning to FIG. 24, another embodiment of the current invention 700 is shown. This embodiment is identical to that shown in FIG. 23 with the exception of the aspiration gas source. In this embodiment the aspiration lumen coupler 726 is attached to a small cylinder of compressed gas 732 via a gas lumen 734 rather than the gas source available in the operating room. This is a convenience to the surgeon as there is one less line connecting to the proximal end 718 of the shaver handle which allows more freedom of movement of the shaver.

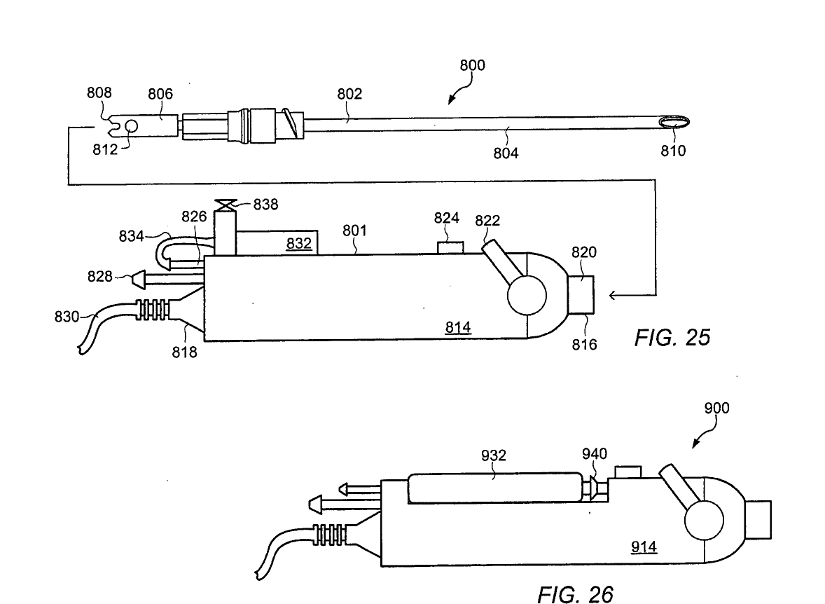

Now turning to FIG. 25, another embodiment of the current invention 800 is shown. This embodiment is identical to that shown in FIG. 24 with the addition of a gas pressure regulator 836. In this embodiment a gas pressure regulator 836 is located between the gas lumen 834 and the gas cylinder 832. The pressure regulator 836 has an adjustment valve 838 which allows the surgeon to control the amount of pressure that is used to aspirate the clogged tissue. With the invention such as that shown in FIG. 23 connected to a gas source in the operating room, there would also be a pressure regulator associated with that gas source located in the operating room which would allow the surgeon to control the aspiration pressure. In the embodiment shown in FIG. 25 with a self-contained gas cylinder the addition of a pressure regulator on the shaver handle allows the surgeon to maintain the same control of the aspiration pressure without the addition of an additional line on the proximal end 818 of the shaver handle.

It will be noted that the type of gas used for aspiration can be any of a number of gases already in common use in the operating room such as but not limited to compressed air, carbon dioxide and nitrogen.

Another embodiment of the current invention 900 is shown in FIG. 26. In this embodiment the gas cylinder 932, is integrated into the shaver handle 914 rather than being attached to the side. The gas cylinder is connected to the handle through the cylinder coupler 940 rather than the gas lumen. This embodiment can also have an optional pressure regulator (not shown) as discussed in FIG. 25. As was discussed with the embodiments shown in FIGS. 24 and 25, this embodiment has the previous advantages of one less proximal line as well as being contained in a similar size and shape as currently available tissue shaving systems.

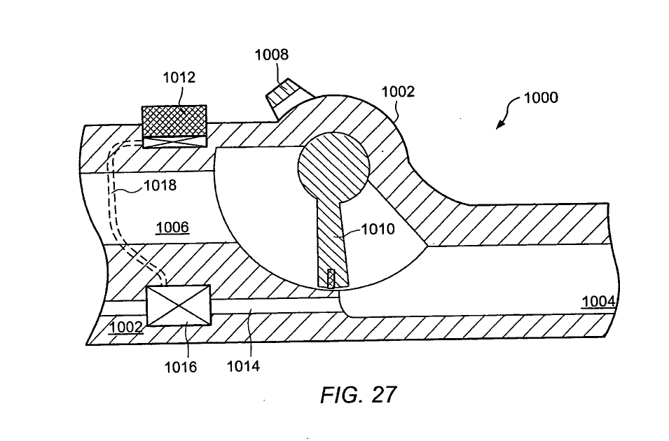

FIG. 27 shows a portion of the cross-section of the shaver handle 1002 of the current invention 1000. Within the shaver handle 1002 are a distal suction/irrigation lumen 1004 and proximal suction/irrigation lumen 1006. The suction flow control actuator 1008 is attached to the suction flow interrupt means 1010 which is shown in the flow interrupt position. In this position flow from the distal suction lumen 1004 to the proximal lumen 1006 is prevented. Also shown are the aspiration flow actuator 1012 which is connected to the aspiration opening means 1016 via connection means 1018 (shown as dashed lines). When the aspiration flow means is activated the opening means 1016 allows pressurized gas to pass through the aspiration lumen 1014 and into the distal suction lumen 1004. The suction interrupt means 1010 contains this pressurized flow and forces it distally towards the clogged tissue in the shaver tip (not shown) forcing the tissue out and unclogging the tip.

The aspiration flow actuator 1012 shown is an electrical switch which operates the aspiration opening means 1016 which is a mechanical valve. It should be obvious to those practiced in the art that these two parts can be combined into a single mechanical valve which would allow pressurized gas to flow into the distal suction lumen.

Although the previously described embodiments all had mechanical tissue resecting distal ends (commonly referred to as shavers), the current invention is equally as beneficial when used with basic suction apparatus that does not have mechanical cutting tips. Basic suction tips are used in most surgery to keep blood or other body fluids or irrigation fluid from accumulating at the operative site. These devices are nominally comprised of a long tube with a distal opening that is placed at the operating site through which excess fluid is removed. Just as with the mechanical cutting tips basic suction devices are attached to a standard vacuum line that is already part of the operating room equipment. Also, just as with the mechanical cutting tips, basic suction device often clog when a piece of debris or tissue that is larger than the distal opening becomes lodged in said distal opening.

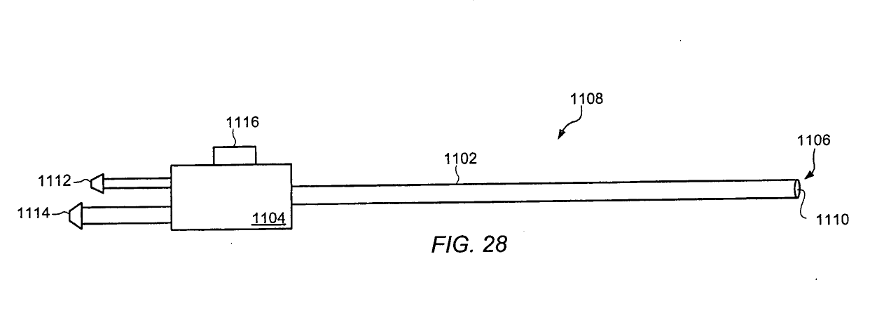

FIG. 28 shows yet another embodiment of the current invention 1100 used for basic surgical fluid removal. The device has a suction tube 1102 and a handle 1104. The distal end 1106 of the suction tube 1102 has an opening 1110 into which fluid is drawn. The proximal end 11011 of the handle 1104 has a suction lumen coupler 1114 as well as an aspiration coupler 1112. Both couplers can be selectably in communication with the suction lumen inside the suction tube 1102 using the aspiration activator means 1116.

In normal operation the suction lumen coupler 1114 which is attached to the vacuum source (not shown) already present in the operating room is in direct communication with the suction tube 1102 such that excess fluid can be drawn into the distal opening 1110 through the tube 1102 out the coupler 1114. When tissue or debris is caught in the distal opening 1110 thereby preventing the flow of excess fluid, the operator can activate the aspiration activator means 1116 which will interrupt the suction flow between the suction lumen coupler 1114 and the distal tube 1102 and then connect the aspiration lumen coupler 1112 to the distal tube allowing the pressured gas source to force the caught tissue distally out of the opening 1110. It will be obvious to those skilled in the art that these two functions of flow interruption followed by aspiration introduction can be combined in the single actuator as described or separated into two actuators. Further, it will also be obvious that these actions can be accomplished either with a direct mechanical valve or indirectly with an electrical switch that operates a mechanical valve. Finally, as was described in FIGS. 24-26, the aspiration means for this basic surgical fluid removal device can also be located in or on the handle 1104 of the device via a gas cylinder rather than from a vacuum source via the aspiration coupler 1112.

Further Improvements

For the embodiments described below, term “fluid” as used herein generally refers to any suitable gas, such as nitrogen, or liquid although use of a gas may be beneficial when practicing some aspects of the presently described embodiments.

The tissue cutting device may include a flush lumen coupled to a source of pressurized gas. The source of pressurized gas may be provided in any suitable manner such as, by way of non-limiting example, a compressed gas bottle, a disposable can or a disposable gas cartridge. The flush lumen may be coupled to an aspiration lumen so that the gas may be used to clear the aspiration lumen of obstructing material.

A valve may be positioned along the flush lumen. The valve is normally in the closed position, and is opened upon actuation of a flush actuator by the user. When the user actuates the flush actuator, the valve opens to permit pressurized gas to pass through the flush lumen and into the aspiration lumen. The valve automatically closes after a short period of time so that the treated area, such as a joint, is not overly inflated. To this end, the valve may be open for less than 0.5 second, or less than 0.3 second. In other words, the volume of gas delivered may be less than 500 ml, or less than 300 ml. Of course, the gas may be delivered for a longer time or larger volumes of gas may be delivered without departing from various aspects of the presently described embodiments. The valve may be any suitable valve familiar to practitioners of ordinary skill in the art, such as a momentary pulsation valve. A gas holding chamber is positioned between the valve and the source of pressurized gas. The gas holding chamber may be expandable so that the chamber expands when filled with the pressurized gas. The chamber may be formed from any suitable materials such as a length of elastic tubing. The chamber may formed from elastic materials or rigid materials which deflect or otherwise displace to increase and decrease in volume as necessary. A flow restrictor may be positioned between the source of pressurized gas and the gas holding chamber. The flow restrictor may be 5-10 times smaller than the smallest dimension of the flush lumen so that the chamber discharges gas relatively quickly but is filled with gas from the source of pressurized gas relatively slowly. The flow restrictor may be, without limitation, any suitable art-recognized flow element including a fixed flow element. An advantage of using a fixed flow control element is that the source of pressurized gas may be a relatively inexpensive compressed gas container with little or no gas pressure regulation. Of course, various aspects of the presently described embodiments may be practiced using an adjustable regulator rather than the restrictor without departing from the invention.

### EXAMPLES

The following will serve to illustrate, by way of one or more examples, systems and methods for inhibiting, reducing or otherwise disrupting prolactin signaling in pain neurons according to some embodiments. The examples below are non-limiting and are intended to be merely representative of various aspects and features of certain embodiments. Although methods and materials similar or equivalent to those described herein may be used in the application or testing of the present embodiments, suitable methods and materials are described below.

### Example 1

A setup using a conventional high pressure (˜2000 psi plus) nitrogen tank and a regulator combined was used to simulate various pressure vessels of 45 psi and

higher. The output of the regulator was connected to a selected length of elastic polyurethane having calculable and modifiable volume to represent a capacitance element. The regulator acts a flow restrictor (that can be a fixed capillary or filter, or a adjustable regulator) that limits the refill speed of the tubing after it has been opened and depressurized momentarily to release the desired bolus of compressed gas for purging the shaver cannula Using flow restrictor controls the refill and re-pressurization of the tubing acting as a capacitor so as to produce a reservoir that is slow to refill but can be discharged rapidly by a push button momentatry-openable valve having considerably less flow restriction (5× or greater) than the fixed flow restrictor or regulator. The rapidly discharged nitrogen or other gas, of volume ranging typically from 50-200 ml exerts sufficient and sudden force to dislodge shaved debris from the tip of the cannula, without releasing excessive gas flow that would overfill the joint being treated, potentially distending and damaging the joint capsule or introducing such a large volume of air that the procedure is slowed by waiting to remove it through the aspiration tube. The air pulse deliver setup was connected to a Stryker shaver.

SUMMARIZED RESULTS: When tested with fragments of clay, elastomer, and human meniscus lodged the cutter-cannula, air pulses at 3040 psi lasting 100-200 ms successfully dislodged the simulated and actual debris clogs, expelling them from the tip of the cannula.

CONCLUSION: The presently described embodiments enable controlled delivery of bolus of purified, sterile inert gas such as nitrogen or other gas. The gas bolus is metered and delivered to the proximal end (housed within the shaver handle) to dislodge cartilage or meniscus or other shaved debris that, from time to time during the procedure, clogs the cutter end of the shaver cannula

This present embodiments cost-effectively and reliably provide a compact mechanism to meter compressed gas to deliver an short, abrupt bolus of valved gas, which is delivered very close to the proximal (within the shaver handle) end of the cannula. These properties provides advantages that enable reliable expulsion of clogging material from the distal end of a tissue shaver during the use thereof in a surgical procedure.
1). The gas bolus is still expanding as it enters the cannula, improving debris expulsion.
2). The gas bolus typically has essentially incompressible fluid in front of it out to the end of the cannula, so that the expulsive force and velocity is efficiently transferred to the debris at the cannula tip.
3). Valving the compressed gas from volume between the flow restrictor and valve (the pneumatic capacitor created by this invention) when communicated through a relatively large passageway, as is done in this invention, results in a brief, high-intensity gas bolus.
   Performance of prior art renditions of the presently disclosed embodiments, on which the embodiments described herein are based has not been fully satisfactory for the purposes described above, such renditions consisting of manual compression of bladders or air compartments, by means of by hand manipulation squeezing a compartment so as to reduce its volume and expel air.

Creating a short, intense gas bolus without the benefit of explosive combustion or a moving piston, using only: a pressurized gas storage vessel; a gas “capacitor” (including a flow restrictor downstream from the gas storage vessel and downstream from the flow restrictor; either an expandable tube or other compartment, or a compartment of sufficient volume to allow expansion of a desired volume of gas to produce the bolus during momentary opening of a valve); and a momentary valve capable of rapidly providing a low restriction communication path to the proximal end of the shaver cannula (located in the drive handle of the shaver), thereby creating a low frequency sonic pulse and shock wave with that suddenly released gas bolus that can travel ahead of the gas/fluid flow to assist in dislodging debris from the shaver cannula tip.

The present invention has been described in connection with a preferred embodiment, however, it is understood that various modifications may be used without departing from the scope of the preceding description.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made in the elements described herein without departing from the spirit and scope of the invention as described in the following claims. In addition, it is to be understood that features described herein independently may, in certain embodiments, be combined.

What is claimed is:

1. A method of cutting tissue comprising the steps:
providing a tissue cutting device comprising an aspiration lumen, a cutting element, a flush lumen and a flush actuator, the flush lumen being coupled to the aspiration lumen;
coupling the aspiration lumen to a suction source;
coupling the flush lumen to a source of pressurized gas;
cutting tissue with the cutting element of the tissue cutting device;
removing tissue which has been cut by the cutting element by drawing the tissue into the aspiration lumen using suction created by the suction source, the tissue being drawn proximally through the aspiration lumen; and
delivering a gas distally through the aspiration lumen to dislodge tissue lodged in the tissue cutting device, the delivering step being carried out upon actuation of the flush actuator by the user.

2. The method of claim 1, wherein the providing step is carried out with a valve positioned along the flush lumen; and the delivering step being carried out with the valve moving from a closed position to an open position and back to the closed position upon actuation of the flush actuator.

3. The method of claim 2, wherein the delivering step is carried out with the valve automatically moving from the closed position to the open position and back to the closed position in less than 0.5 second.

4. The method of claim 3, wherein the delivering step is carried out with the valve moving back to the closed position in less than 0.3 second.

5. The method of claim 2, wherein the providing step is carried out with a gas holding chamber positioned between the valve and the source of pressurized gas, the gas holding chamber receiving gas from the source of pressurized gas; and the delivering step being carried out with the gas in the gas holding chamber being delivered through the flush lumen

6. The method of claim 5, wherein the delivering step is carried out with a volume of gas delivered to the aspiration lumen being less than a volume of gas contained between the valve and the source of pressurized gas.

7. The method of claim 5, wherein the providing step is carried out with the gas holding chamber being expandable; and the delivering step being carried out with the gas holding chamber reducing a volume when gas is discharged from the gas holding chamber.

8. The method of claim 1, wherein the delivering step being carried out with a volume of gas delivered being less than 500 ml.

9. The method of claim 7, wherein the providing step is carried out with a flow restrictor positioned between the gas holding chamber and the source of pressurized gas, the flow restrictor providing direct pressure communication between the gas holding chamber and the source of pressurized gas while limiting a flow rate of the gas from the source of pressurized gas into the gas holding chamber.

10. The method of claim 1, wherein the providing step is carried out with the tissue cutting device having an outer tube and an inner tube, the inner tube being positioned within the outer tube and the aspiration lumen extending through the inner tube, the cutting element being positioned at a distal end of the inner tube.

11. A system for cutting tissue, comprising:
a body;
a tissue cutting element coupled to the body;
an aspiration lumen coupled to the body, the aspiration lumen having an opening positioned to draw tissue cut by the cutting element into the aspiration lumen;
a suction source coupled to the aspiration lumen;
a flush lumen coupled to the aspiration lumen;
a source of pressurized gas coupled to the flush lumen; and
a flush actuator having an actuation position, the flush actuator permitting gas to pass from the flush lumen into the aspiration lumen when moved to the actuation position.

12. The system of claim 11, further comprising:
a valve positioned on the flush lumen;
the flush actuator causing the valve to automatically move from a closed position to an open position and back to the closed position, the valve permitting the gas to pass through the flush lumen and into the aspiration lumen when the valve is in the open position.

13. The system of claim 12, wherein the valve automatically moves from the closed position to the open position and back to the closed position in less than 0.5 second.

14. The system of claim 13, wherein the valve moves back to the closed position in less than 0.3 second.

15. The system of claim 14, further comprising a gas holding chamber positioned between the valve and the source of pressurized gas, the gas holding chamber receiving gas from the source of pressurized gas.

16. The system of claim 15, wherein the gas holding chamber is expandable, the gas holding chamber at least partially collapsing when gas is delivered through the flush lumen and at least partially expanding when gas is delivered from the source of pressurized gas.

17. The system of claim 16, further comprising a flow restrictor positioned between the gas holding chamber and the source of pressurized gas, the flow restrictor providing direct pressure communication between the gas holding chamber and the source of pressurized gas while limiting a flow rate of the gas into the gas holding chamber so that the pressurized gas enters the gas holding chamber at a rate lower than the gas leaves the gas holding chamber when the flush actuator is moved to the actuation position.

18. The system of claim 11, wherein the valve is configured to permit less than 500 ml of the pressurized gas to pass through the valve when the flush actuator is actuated.

19. The system of claim 11, wherein the body includes an outer tube and an inner tube, the inner tube being positioned within the outer tube and the aspiration lumen extending through the inner tube, the cutting element being positioned at a distal end of the inner tube.
